(19) United States
(12) Patent Application Publication
(10) Pub. No.: US 2010/0197924 A1
(43) Pub. Date: Aug. 5, 2010

(54) PREPARATION OF AMINOTETRALIN COMPOUNDS

(75) Inventors: Alexandra E. Gould, Cambridge, MA (US); Sean J. Harrison, Belmont, MA (US); Hirotake Mizutani, Cambridge, MA (US); Ming Shen, Winchester, MA (US); Thomas E. Smyser, Franklin, MA (US); Stephen G. Stroud, Medford, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc., Cambridge, MA (US)

(21) Appl. No.: 12/639,239

(22) Filed: Dec. 16, 2009

Related U.S. Application Data
(60) Provisional application No. 61/203,419, filed on Dec. 22, 2008.

(51) Int. Cl. C07D 471/04; C07C 211/38; C07C 209/26; C07C 69/76; C07C 67/24; C07C 229/54; C07C 227/04; C07C 67/00; C07D 233/64; C07D 231/12

(52) U.S. Cl. 546/123; 564/307; 560/51; 562/449; 560/38; 562/444; 548/343.5; 548/377.1

(57) ABSTRACT

The present invention relates to synthetic processes for preparation of aminotetralin compounds with kinase inhibitory activity. The invention also provides synthetic intermediates useful in the processes of the invention.

The present application claims the benefit of U.S. Provisional Application Ser. No. 61/203,419, filed Dec. 22, 2008 (pending). The entire contents of U.S. Provisional Application Ser. No. 61/203,419 is incorporated herein by this reference.

### BACKGROUND OF THE INVENTION

1. Field of the Invention

The present invention relates to synthetic processes for preparation of aminotetralin compounds with kinase inhibitory activity. The invention also provides synthetic intermediates useful in the processes of the invention.

2. Background of the Invention

Intracellular signaling pathways activated in response to growth factor/cytokine stimulation are known to control functions such as proliferation, differentiation and cell death (Chiloeches and Marais, In Targets for Cancer Therapy; Transcription Factors and Other Nuclear Proteins, 179-206 (La Thangue and Bandara, eds., Totowa, Humana Press 2002)). One example is the Ras-Raf-MEK-ERK pathway which is controlled by receptor tyrosine kinase activation. Activation of Ras proteins at the cell membrane leads to phosphorylation and recruitment of accessory factors and Raf which is then activated by phosphorylation. Activation of Raf leads to downstream activation of MEK and ERK. ERK has several cytoplasmic and nuclear substrates, including ELK and Ets-family transcription factor, which regulates genes involved in cell growth, survival and migration (Marais et al., J. Biol. Chem., 272:4378-4383 (1997); Peyssonnaux and Eychene, Biol. Cell, 93-53-62 (2001)). As a result, this pathway is an important mediator of tumor cell proliferation and angiogenesis. For instance, overexpression of constitutively active B-Raf can induce an oncogenic event in untransformed cells (Wellbrock et al., Cancer Res., 64:2338-2342 (2004)). Aberrant activation of the pathway, such as by activating Ras and/or Raf mutations, is known to be associated with a malignant phenotype in a variety of tumor types (Bos, Hematol. Pathol., 2:55-63 (1988); Downward, Nature Rev. Cancer, 3:11-22 (2003); Karasarides et al., Oncogene, 23:6292-6298 (2004); Tuveson, Cancer Cell, 4:95-98 (2003); Bos, Cancer Res, 49:4682-4689 (1989)). Activating mutations in B-Raf are found in 60-70% of melanomas. Melanoma cells that carry mutated B-Raf-V600E are transformed, and cell growth, ERK signaling and cell viability are dependent on mutant B-Raf function (Karasarides et al., Oncogene, 23:6292-6298 (2004)).

Inhibitors of the Raf kinases may be expected to interrupt the Ras-Raf signaling cascade and thereby provide new methods for the treatment of proliferative disorders, such as cancer. U.S. Ser. No. 07/0,149,533 reports aminotetralin compounds with Raf kinase inhibitory activity. The compounds are useful for inhibiting Raf kinase activity in vitro and in vivo. The compounds also are useful for inhibiting cell proliferation, and are particularly useful for the treatment of various cell proliferative diseases. There is thus a need for improved synthetic procedures for preparing such aminotetralin compounds.

### DESCRIPTION OF THE INVENTION

The present invention provides synthetic processes useful for preparing aminotetralin compounds. The invention also provides synthetic intermediates useful in the processes of the invention.

In a first aspect, the invention provides a process for preparing a compound of formula (I):

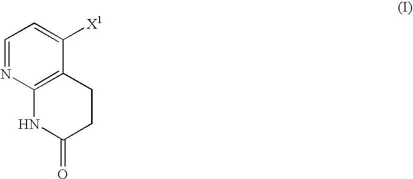

wherein X¹ is Cl or F.

The process comprises treating a compound of formula (II):

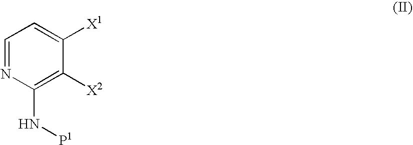

wherein X¹ is Cl or F, X² is Br or I, and P¹ is hydrogen or an amino group protecting moiety that is labile to the reaction conditions;

with a compound of formula (III):

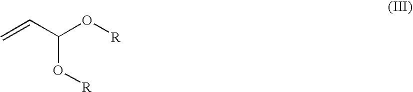

wherein each R independently is C₁₋₄ alkyl, —C(O)—(C₁₋₄ alkyl), C₆₋₁₀ ar(C₁₋₄)alkyl, or —C(O)—(C₆₋₁₀ ar(C₁₋₄)alkyl), where the aryl portion of any such groups is substituted or unsubstituted;
in a reaction mixture comprising a palladium catalyst and a base, to form the compound of formula (I).

In the compounds of formulae (I)-(II), X¹ is Cl or F, and X² is Br or I. In some embodiments X¹ is F. In some embodiments, X² is 1. In certain embodiments, X¹ is F and X² is I.

In the compounds of formula (III), each R independently is C₁₋₄ alkyl, —C(O)—(C₁₋₄ alkyl), C₆₋₁₀ ar(C₁₋₄)alkyl, or —C(O)—(C₆₋₁₀ ar(C₁₋₄)alkyl), where the aryl portion of any such groups is substituted or unsubstituted. In some embodiments, each R is C₁₋₄ alkyl or C₆₋₁₀ ar(C₁₋₄)alkyl, where the aryl portion is substituted or unsubstituted. In certain embodiments, each R is methyl, ethyl, or benzyl. In certain particular embodiments, each R is methyl or ethyl.

The coupling reaction of a compound of formula (II) with a compound of formula (III) is effected in the presence of a palladium catalyst and a base. Palladium catalysts suitable for use in the coupling reaction include those generally known in the art to be useful in Heck reactions. (Brase, Stefan; De Meijere, Armin. Metal-Catalyzed Cross-Coupling Reactions, (1998), 99-166). In some embodiments, the palladium catalyst is selected from the group consisting of palladium(II) chloride, palladium(II) acetate, tris(dibenzylideneacetone)-dipalladium, tetrakis(triphenylphosphine)palladium, bis(triphenylphosphine)palladium dichloride, (1,1′-bis(diphenylphosphino)ferrocene)palladium dichloride, di-chlorobis[5-chloro-2-[(4-chlorophenyl)(hydroxyimino)methyl]phenyl-C]di-palladium (Najéra's catalyst), and trans-di-μ-acetobis [2-(di-o-tolylphosphino)benzyl]dipalladium (Hermann's catalyst).

In some embodiments, the reaction mixture further comprises an added phosphine ligand. An added phosphine ligand is particularly advantageous in those embodiments wherein the palladium catalyst is selected from the group consisting of palladium(II) chloride, palladium(II) acetate, tris(dibenzylideneacetone)dipalladium, bis(triphenylphosphine)palladium dichloride, (1,1′-bis(diphenylphosphino)ferrocene)palladium dichloride. Nonlimiting examples of suitable ligands include triphenylphosphine, tri(o-tolyl)-phosphine, tri(tert-butyl)phosphine, tri(2-furyl)phosphine, 1,1′-bis(diphenylphosphino)-ferrocene, 1,1′-bis(diphenylphosphino)methane, 1,1′-bis(diphenylphosphino)ethane, and 2-dicyclohexylphosphino-2′,4′,6′-triisopropylbiphenyl (XPhos).

In certain particular embodiments, the palladium catalyst is di-chlorobis[5-chloro-2-[(4-chlorophenyl)(hydroxyimino)methyl]phenyl-C]di-palladium or trans-di-μ-acetobis-2-(di-o-tolylphosphino)benzyl]dipalladium. In certain other particular embodiments, the palladium catalyst is palladium(II) acetate, and the reaction mixture further comprises tri(o-tolyl)phosphine. In certain other particular embodiments, the palladium catalyst is palladium(II) acetate, and the reaction mixture does not comprise an added phosphine ligand.

The reaction mixture also comprises a base. In some embodiments, the base is selected from the group consisting of potassium carbonate, cesium carbonate, sodium carbonate, sodium bicarbonate, sodium hydroxide, potassium hydroxide, lithium hydroxide, sodium acetate, and potassium acetate. In some other embodiments, the base is a tertiary amine base. In some such embodiments, the tertiary amine base is selected from the group consisting of triethylamine, diisopropylethylamine, dicyclohexylmethylamine, and 1,8-diazabicyclo-[5.4.0]undec-7-ene.

The reaction mixture typically also comprises a solvent. In various embodiments, the solvent has a boiling point above 90° C., above 100° C., above 110° C., above 120° C., or above 130° C. In some embodiments, the coupling reaction of a compound of formula (II) with a compound of formula (III) is conducted in a solvent comprising dimethylformamide (DMF), dimethylacetamide (DMA), N-methylpyrrolidone (NMP), 1,4-dioxane, tert-butanol, or a mixture thereof. In some embodiments, the solvent also comprises water. In certain embodiments, the solvent comprises dimethylformamide-water or dimethylacetamide-water.

The coupling reaction preferably is conducted at elevated temperature. In some embodiments, the reaction mixture is heated at a temperature in the range of about 100° C. to about 170° C. In some embodiments, the reaction mixture is heated at a temperature in the range of about 120° C. to about 150° C.

In the compounds of formula (II), P is hydrogen or an amino group protecting moiety. When P is an amino group protecting moiety, it must be labile to the reaction conditions in order for cyclization of the lactam to occur. In some embodiments, P is tert-butoxycarbonyl. In such embodiments, the solvent preferably includes water to effect hydrolysis of P under the reaction conditions. Without wishing to be bound by theory, applicants believe that the tert-butoxycarbonyl group is removed from the compound of formula (II) prior to coupling with the compound of formula (III).

In some embodiments, the process of the invention further comprises preparation of the compound of formula (II). In some such embodiments, the process further comprises the steps:
(aa) treating a compound of formula (IV):

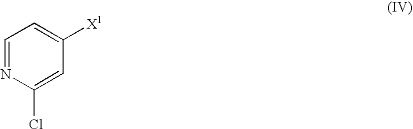

wherein X¹ is Cl or F;
with a compound of formula P¹—NH₂, wherein P¹ is an amino group protecting moiety, in a reaction mixture comprising a palladium catalyst and a base to form a compound of formula (V):

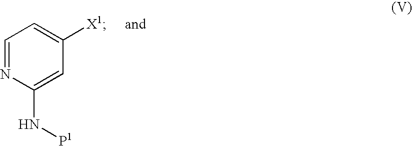

(bb) halogenating the compound of formula (V) to form the compound of formula (II); wherein P is an amino group protecting moiety.

Step (aa) preferably is conducted under conditions known in the art to be effective for Hartwig-Buchwald coupling reactions. (Hartwig, J. F, Angew. Chem. Int. Ed., 1998, 37, 2046-2067). In some embodiments, the palladium catalyst in step (aa) is selected from the group consisting of palladium(II) chloride, palladium(II) acetate, tris(dibenzylideneacetone)-dipalladium, tetrakis(triphenylphosphine)palladium, bis(triphenylphosphine)palladium dichloride, and (1,1′-bis(diphenylphosphino)ferrocene)palladium dichloride.

In some embodiments, the reaction mixture in step (aa) further comprises an added phosphine ligand. Nonlimiting examples of suitable ligands include tri(o-tolyl)phosphine, triphenylphosphine, tri(2-furyl)phosphine, 1,1′-bis(diphenylphosphino)-ferrocene, 1,1′-bis(diphenylphosphino)methane, 1,1′-bis(diphenylphosphino)ethane, (oxydi-2,1-phenylene)bis(diphenylphosphine), 4,5-bis(diphenylphosphino)-9,9-dimethylxanthene (xantphos), 2-dicyclohexylphosphino-2′,6′-dimethoxybiphenyl (SPhos), and 2-dicyclohexylphosphino-2′,4′,6′-triisopropylbiphenyl (XPhos). In certain embodiments, the palladium catalyst is palladium(II) acetate, and the phosphine ligand is xantphos, Xphos, or (oxydi-2,1-phenylene)bis(diphenylphosphine).

In some embodiments, the base employed in step (aa) is selected from the group consisting of potassium carbonate, cesium carbonate, sodium carbonate, sodium bicarbonate, sodium tert-butoxide, potassium tert-butoxide, sodium hydroxide, potassium hydroxide, and lithium hydroxide.

Preferably the coupling reaction (aa) is conducted in a high-boiling solvent. In some embodiments, the coupling reaction of a compound of formula (IV) is conducted in a solvent comprising dimethylformamide, dimethylacetamide, N-methylpyrrolidone (NMP), 1,4-dioxane, isopropanol, tert-butanol, toluene, benzene, or a mixture thereof. In certain embodiments, the solvent is 1,4-dioxane or toluene.

The coupling reaction of step (aa) preferably is conducted at elevated temperature. In some embodiments, the reaction mixture is heated at a temperature in the range of about 70° C. to about 150° C. In some embodiments, the reaction mixture is heated at a temperature in the range of about 80° C. to about 110° C.

In some embodiments, the halogenating step (bb) comprises treating the compound of formula (V) with a base and iodine to form the compound of formula (II) wherein X² is I, and P is an amino group protecting moiety. In some such embodiments, the base is an organolithium, an organomagnesium, or a silver salt. In certain embodiments, the base is selected from the group consisting of tert-butyllithium, n-butyllithium, di-n-butylmagnesium and silver sulfate.

In some other embodiments, the halogenating step (bb) comprises treating the compound of formula (V) with a base and a brominating reagent to form the compound of formula (II), wherein X² is Br, and P is an amino group protecting moiety. In some such embodiments, the brominating reagent is selected from the group consisting of ethylene bromide, N-bromosuccinimide, and bromine. In some such embodiments, the base is an organolithium, an organomagnesium, or a silver salt In certain embodiments, the base is selected from the group consisting of tert-butyllithium, n-butyllithium, di-n-butylmagnesium and silver sulfate.

When an organolithium is used in the halogenating step (bb), the reaction mixture may additionally comprise a ligand that complexes lithium. Nonlimiting examples of such complexing ligands include tetrahydrofuran (THF), tetramethylethylenediamine (TMEDA), hexamethylphosphoramide (HMPA), and 1,4-diazabicyclo[2.2.2]octane (DABCO).

In some other embodiments, the halogenating step (bb) comprises treating the compound of formula (V) with a tertiary amine and bromine to form the compound of formula (II), wherein X² is Br, and P is an amino group protecting moiety. In some such embodiments, the tertiary amine is triethylamine.

In some embodiments, the compound of formula (II) formed by steps (aa) and (bb) is used directly in the coupling reaction with a compound of formula (III). In such embodiments, P¹ is an amino group protecting moiety that is labile to the reaction conditions for coupling the compound of formula (II) with a compound of formula (III).

In other embodiments, preparation of the compound of formula (II) further comprises the step:
(cc) removing the protecting group P¹ to form the compound of formula (II), wherein P is hydrogen.

In such embodiments, the amino group protecting moiety P¹ can be any protecting group that is conveniently removed in step (cc). Non-limiting examples of amino group protecting moieties can be found in P. G. M. Wuts and T. W. Greene, Greene's Protective Groups in Organic Synthesis (4^{th} ed.), John Wiley & Sons, NJ (2007), and include, e.g., acyl, sulfonyl, oxyacyl, and aminoacyl groups.

In another aspect, the invention provides a process for preparing a compound of formula (VI):

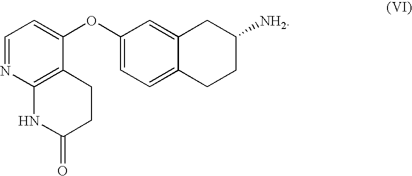

The process comprises the steps:
(i) coupling a compound of formula (I):

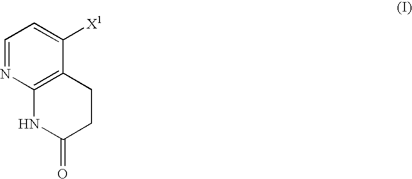

wherein X¹ is Cl or F;

with a compound of formula (VII):

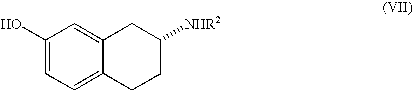

wherein R² is hydrogen, an amino group protecting moiety, or an acid addition salt; to form the compound of formula (VI-A):

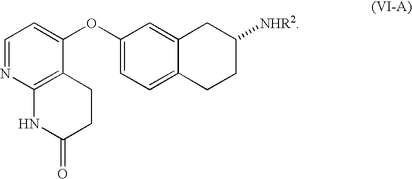

When R² is an amino group protecting moiety, the protecting moiety must be removed to form the compound of formula (VI). The deprotection may occur in situ in the reaction mixture for the coupling reaction, or it may be accomplished in a separate step.

The coupling of a compound of formula (I) with a compound of formula (VII) can be accomplished under a variety of reaction conditions. In some embodiments, the coupling is conducted in a reaction mixture comprising a copper or palladium catalyst. In some other embodiments, the coupling is conducted in a reaction mixture comprising a base and a high-boiling, polar solvent. Suitable bases include, without limitation, cesium carbonate, potassium carbonate, potassium hydroxide, potassium-tert-butoxide, sodium-tert-butoxide, sodium methoxide, potassium methoxide, and sodium hydride. Suitable solvents include, without limitation, dimethylformamide, dimethylacetamide, dimethyl sulfoxide, and 1,4-dioxane. In some embodiments, the reaction mixture comprises cesium carbonate and dimethylformamide. Preferably, the coupling reaction is conducted at elevated temperature. In some embodiments, the reaction mixture is heated at a temperature in the range of about 80° C. to about 260° C. In some embodiments, the reaction mixture is heated at a temperature in the range of about 90° C. to about 160° C. In certain embodiments, the reaction mixture is heated at a temperature in the range of about 140° C. to about 150° C. In some embodiments, microwave irradiation is utilized to facilitate the reaction.

In some embodiments, R² is an amino group protecting moiety. In some such embodiments, R² is an oxyacyl moiety. In certain such embodiments, R² is tert-butoxycarbonyl. In some other embodiments, R² is an acid addition salt. In certain such embodiments, R² is H.HBr. In some embodiments, R² is selected from the group consisting of hydrogen, tert-butoxycarbonyl, and H.HBr.

In some embodiments, the process further comprises the step:
(iii) condensing the compound of formula (VI) with a compound of formula (VIII):

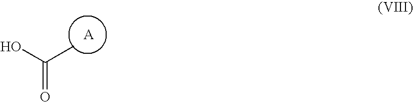

wherein Ring A is a substituted or unsubstituted phenyl ring, to form a compound of formula (IX):

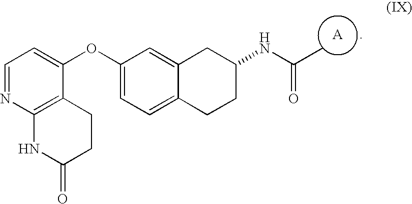

Amide forming reaction conditions suitable for use in the condensing step (iii) are well known in the art.

In some embodiments, the compound of formula (VIII) is characterized by formula (VIII-A):

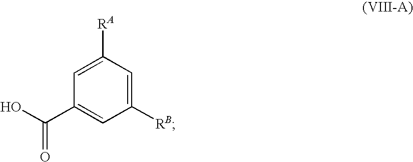

and the compound of formula (IX) is characterized by formula (IX-A):

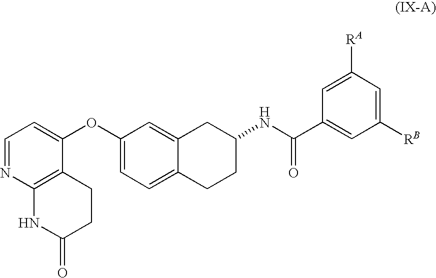

wherein:
R^{A} is halo, —CN, —CHO, —C(R^{5x})═C(R^{5x})(R^{5y}), —C≡C—R^{5Y}, —OR^{5z}, —N(R^{4y})(R^{4z}), —CO₂R^{6x}, —C(O)N(R^{4x})(R^{4y}); or R^{A} is a C₁₋₆ aliphatic or C₁₋₆ fluoroaliphatic optionally substituted with one or two substituents independently selected from the group consisting of —OR^{5z}, —N(R^{4y})(R^{4z}), —SR^{6x}, —CO₂R^{6x}, or —C(O)N(R^{4x})(R^{4y}); or R^{A} is an optionally substituted 5- or 6-membered nitrogen-containing heterocyclyl or heteroaryl ring;
R^{B} is selected from the group consisting of C₁₋₄ aliphatic, C₁₋₄ fluoroaliphatic, —O(C₁₋₄aliphatic), —O(C₁₋₄fluoroaliphatic), and halo; and
R^{4x} is hydrogen, C₁₋₄ aliphatic, C₁₋₄ fluoroaliphatic, or C₆₋₁₀ ar(C₁₋₄)alkyl, the aryl portion of which may be optionally substituted;
R^{4y} is hydrogen, C₆₋₁₀ ar(C₁₋₄)alkyl, the aryl portion of which may be optionally substituted, an optionally substituted 5- or 6-membered aryl, heteroaryl, or heterocyclyl ring, or a C₁₋₄ aliphatic or C₁₋₄ fluoroaliphatic optionally substituted with one or two substituents independently selected from the group consisting of —OR^{5x}, —N(R^{4x})₂, —CO₂R^{5x}, or —C(O)N(R^{4x})₂;
R^{4z} is an amino group protecting moiety, C₁₋₄ aliphatic, C₁₋₄ fluoroaliphatic, or C₆₋₁₀ ar(C₁₋₄)alkyl, the aryl portion of which may be optionally substituted; or
R^{4x} and R^{4y}, taken together with the nitrogen atom to which they are attached, form an optionally substituted 4- to 8-membered heterocyclyl or 5-membered heteroaryl ring having, in addition to the nitrogen atom, 0-2 ring heteroatoms independently selected from N, O, and S; or
R^{4y} and R^{4z}, taken together with the nitrogen atom to which they are attached, form an optionally substituted 4- to 8-membered heterocyclyl or 5-membered heteroaryl ring having, in addition to the nitrogen atom, 0-2 ring heteroatoms independently selected from N, O, and S;
each R^{5x} independently is hydrogen, C₁₋₄ aliphatic, C₁₋₄ fluoroaliphatic, or C₆₋₁₀ ar(C₁₋₄)alkyl, the aryl portion of which may be optionally substituted, or an optionally substituted 5- or 6-membered aryl, heteroaryl, or heterocyclyl ring;
each R^{5y} independently is hydrogen, an optionally substituted monocyclic nitrogen-containing heterocyclyl, an optionally substituted C₆₋₁₀ aryl, a C₆₋₁₀ar(C₁₋₄)alkyl, the aryl portion of which is optionally substituted, or a C₁₋₄ aliphatic or C₁₋₄ fluoroaliphatic optionally substituted with one or two substituents independently selected from the group consisting of —OR^{5x}, —N(R^{4x})₂, —CO₂R^{5x}, or —C(O)N(R^{4x})₂;
each R^{5z} independently is hydrogen, a hydroxy group protecting moiety, an optionally substituted monocyclic nitrogen-containing heterocyclyl, an optionally substituted C₆₋₁₀ aryl, a C₆₋₁₀ar(C₁₋₄)alkyl, the aryl portion of which is optionally substituted, or a C₁₋₄ aliphatic or C₁₋₄ fluoroaliphatic optionally substituted with one or two substituents independently selected from the group consisting of —OR^{5z}, —N(R^{4x})(R^{4y}), —CO₂R^{6x}, or —C(O)N(R^{4x})(R^{4y}); and

each R^{6x} independently is C₁₋₄ aliphatic, C₁₋₄ fluoroaliphatic, or C₆₋₁₀ ar(C₁₋₄)alkyl, the aryl portion of which may be optionally substituted.

In some such embodiments, R^{A} is a substituted or unsubstituted pyrazolyl, oxazolyl, isoxazolyl, imidazolyl, triazolyl, or tetrazolyl ring. In some embodiments, each substitutable ring carbon atom in R^{A} independently is unsubstituted or is substituted with halo, —OR^{5x}, —N(R^{4x})(R^{4y}), —N(R^{4x})—C(O)—R⁵, —C(O)—N(R^{4x})(R^{4y}), or a C₁₋₄ aliphatic or C₁₋₄ fluoroaliphatic group optionally substituted with ═O, —OR^{5x}, —N(R^{4x})(R^{4y})—C(O)—R⁵, or —C(O)—N(R^{4x})(R^{4y}); and each substitutable ring nitrogen atom in R^{A} is unsubstituted or is substituted with —C(O)—R⁵, —C(O)N(R^{5x})₂, —SO₂—R⁵, or a C₁₋₄ aliphatic or C₁₋₄ fluoroaliphatic group optionally substituted with ═O, —OR^{5x}, —N(R^{4x})(R^{4y}), —N(R^{4x})—C(O)—R⁵, or —C(O)—N(R^{4x})(R^{4y}), where the variables R^{4x}, R^{4y}, R⁵, and R^{5x} have the values described above.

In some other embodiments, R^{A} has the formula —C(R^{a})(R^{b})—N(R^{c})(R^{d}), where:
R^{a} is hydrogen, C₁₋₄ aliphatic, C₁₋₄ fluoroaliphatic, or -T¹-R²; or R^{a}, taken together with R^{b} and the carbon atom to which they are attached, forms a substituted or unsubstituted 3- to 6-membered carbocyclic ring; or R^{a}, taken together with R^{c} and the intervening carbon and nitrogen atoms, form a substituted or unsubstituted 4- to 6-membered heterocyclic ring;
R^{b} is hydrogen, C₁₋₄ aliphatic, or C₁₋₄ fluoroaliphatic; or R^{b}, taken together with R^{a} and the carbon atom to which they are attached, forms a substituted or unsubstituted 3- to 6-membered carbocyclic ring;
R^{c} is hydrogen, C₁₋₄ aliphatic, C₁₋₄ fluoroaliphatic, or -T¹-R²; or R^{c}, taken together with R^{a} and the intervening carbon and nitrogen atoms, form a substituted or unsubstituted 4- to 6-membered heterocyclic ring; or R^{c}, taken together with R^{d} and the nitrogen atom to which they are attached, forms a substituted or unsubstituted 3- to 6-membered heterocyclic ring or 5- to 6-membered heteroaryl ring;
R^{d} is an amino group protecting moiety, C₁₋₄ aliphatic, or C₁₋₄ fluoroaliphatic, or -T¹-R²; or R^{d}, taken together with R^{c} and the nitrogen atom to which they are attached, forms a substituted or unsubstituted 3- to 6-membered heterocyclic ring or 5- to 6-membered heteroaryl ring;
T¹ is a C₁₋₃ alkylene chain;
R² is —OR^{5z}, —N(R^{4y})(R^{4z}), —N(R^{4x})—C(O)—R^{5x}, or —C(O)—N(R^{4x})(R^{4y}); and

the variables R^{4x}, R^{4y}, R^{4z}, R^{5x}, and R^{5z} have the values described above.

In some other embodiments, the compound of formula (VIII) is characterized by formula (VIII-B):

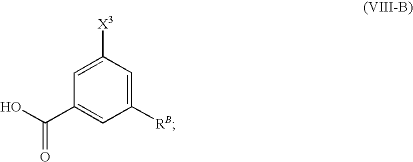

and the compound of formula (IX) is characterized by formula (IX-B):

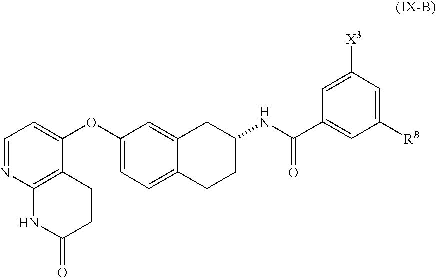

wherein:
X³ is Br or I; and
R^{B} is selected from the group consisting of Cl, C₁₋₄ aliphatic, C₁₋₄ fluoroaliphatic, —O(C₁₋₄ aliphatic), and —O(C₁₋₄ fluoroaliphatic).

In some embodiments, the process further comprises the step:
(iv-a) coupling the compound of formula (IX-B) with a compound of formula (X):

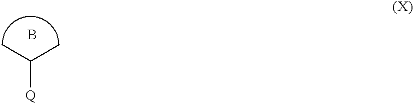

wherein Ring 13 is a substituted or unsubstituted aryl or heteroaryl ring; and
Q is a moiety selected from the group consisting of boronic acid, zinc halide, and trialkyltin;
in a reaction mixture comprising a palladium catalyst, to form a compound of formula (IX-C):

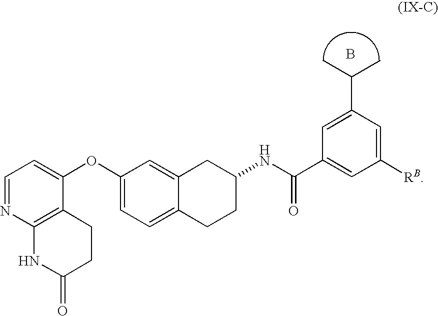

Step (iv-a) preferably is conducted under conditions known in the art to be effective for Suzuki coupling (Herrmann, Wolfgang A. Applied Homogeneous Catalysis with Organometallic Compounds (2nd Edition) 2002, 1 591-598), Stile coupling (Farina, V.; Krishnamurthy, V.; Scott, W. J., Org. React. 1998, 50, 1-652.), or Negishi coupling (Negishi, Ei-ichi; Hu, Qian; Huang, Zhihong; Qian, Mingxing; Wang, Guangwei Aldrichimica Acta 2005, 38, 71-88). Examples of palladium catalysts and phosphine ligands useful for step (iv-a) include those described above for step (a).

In some other embodiments, the process further comprises the step:
(iv-b) coupling the compound of formula (IX-B) with a compound of formula (XI):

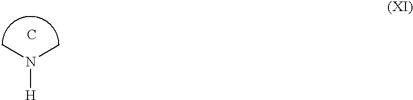

wherein Ring C is a substituted or unsubstituted heteroaryl ring;
in a reaction mixture comprising a copper or palladium catalyst, to form a compound of formula (IX-D):

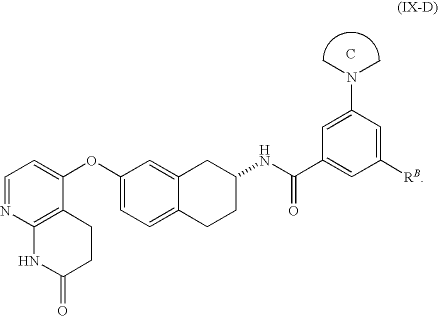

Step (iv-b) preferably is conducted under conditions known to be effective for Hartwig-Buchwald coupling reactions. Examples of palladium catalysts and phosphine ligands suitable for use in step (iv-b) include those described above for step (aa). Step (iv-b) also can be conducted using conditions known to be effective for Ullmann copper catalysed coupling reactions (Elson, Todd D. and Crouch, R. David Organic Reactions, 63:265-555 (2004)).

In some other embodiments, the compound of formula (VIII) is characterized by formula (VIII-C):

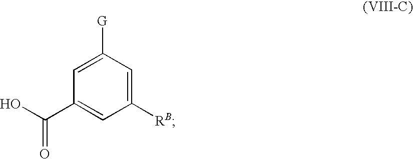

and the compound of formula (IX) is characterized by formula (IX-C):

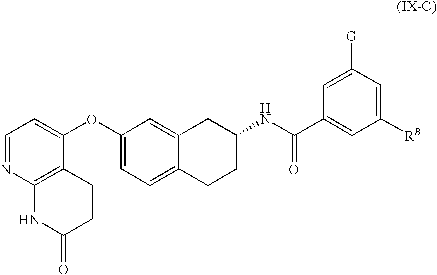

wherein:
G is —CN or —CHO; and
R⁵ is selected from the group consisting of Cl, C₁₋₄ aliphatic, C₁₋₄ fluoroaliphatic, —O(C₁₋₄ aliphatic), and —O(C₁₋₄ fluoroaliphatic).

In some such embodiments, wherein G is —CN, the process further comprises treating the compound of formula (IX-C) with sodium azide to form a compound of formula (IX), wherein R^{A} is tetrazolyl.

In some other such embodiments, wherein G is —CHO, the process further comprises treating the compound of formula (IXC) with p-tolylsulfonylmethyl isocyanide to form a compound of formula (IX), wherein R^{A} is 1,3-oxazol-5-yl.

In some other such embodiments, wherein G is —CHO, the process further comprises treating the compound of formula (IX-C) with p-tolylsulfonylmethyl isocyanide, followed by ammonia or a primary amine, to form a compound of formula (IX), wherein R^{A} is imidazolyl.

In some other such embodiments, wherein G is —CHO, the process further comprises treating the compound of formula (IX-C) with an amine of formula HN(R^{c})(R^{d}) and a reducing agent to form a compound of formula (IX), wherein R^{A} has the formula —CH₂N(R^{c})(R^{d}).

In another aspect, the invention provides novel compounds useful in the processes of the invention. In one embodiment, the invention provides a compound of formula (I) or a salt thereof:

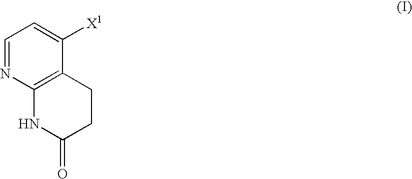

wherein X¹ is Cl or F.

In another embodiment, the invention provides a compound of formula (II) or a salt thereof:

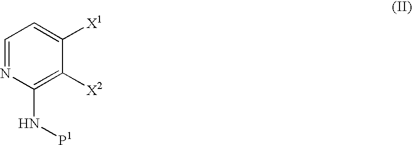

wherein X¹ is Cl or F, X² is Br or I, and P¹ is hydrogen or an amino group protecting moiety.

In yet another embodiment, the invention provides a compound of formula (VI-A):

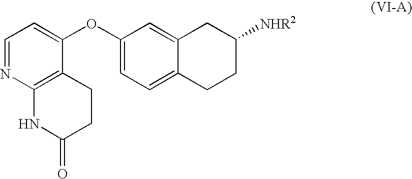

wherein R² is hydrogen, an amino group protecting moiety, or an acid addition salt.

The terms “Raf” and “Raf kinase” are used interchangeably, and unless otherwise specified refer to any member of the Raf family of kinase enzymes, including without limitation, the isoforms A-Raf, B-Raf, and C-Raf. These enzymes, and the corresponding genes, also may be referred to in the literature by variants of these terms, e.g., RAF, raf, BRAF, B-raf. The isoform C-Raf also is referred to by the terms Raf-1 and C-Raf-1.

The term “amino group protecting moiety” refers to any group useful in organic synthesis for protecting an amino group. Preferably, the amino group protecting moiety is conveniently added and removed under conditions that do not interfere with other functional groups in the molecule. Non-limiting examples of amino group protecting moieties can be found in P. G. M. Wuts and T. W. Greene, Greene's Protective Groups in Organic Synthesis (4^{th} ed.), John Wiley & Sons, NJ (2007), and include, e.g., acyl, sulfonyl, oxyacyl, and aminoacyl groups.

The term “aliphatic” or “aliphatic group”, as used herein, means a substituted or unsubstituted straight-chain, branched, or cyclic C₁₋₁₂ hydrocarbon, which is completely saturated or which contains one or more units of unsaturation, but which is not aromatic. For example, suitable aliphatic groups include substituted or unsubstituted linear, branched or cyclic alkyl, alkenyl, or alkynyl groups and hybrids thereof, such as (cycloalkyl)alkyl, (cycloalkenyl)alkyl or (cycloalkyl)alkenyl. In various embodiments, the aliphatic group has 1 to 12, 1 to 8, 1 to 6, 1 to 4, or 1 to 3 carbons.

The terms “alkyl”, “alkenyl”, and “alkynyl”, used alone or as part of a larger moiety, refer to a straight or branched chain aliphatic group having from 1 to 12 carbon atoms. For purposes of the present invention, the term “alkyl” will be used when the carbon atom attaching the aliphatic group to the rest of the molecule is a saturated carbon atom. However, an alkyl group may include unsaturation at other carbon atoms. Thus, alkyl groups include, without limitation, methyl, ethyl, propyl, allyl, propargyl, butyl, pentyl, and hexyl.

For purposes of the present invention, the term “alkenyl” will be used when the carbon atom attaching the aliphatic group to the rest of the molecule forms part of a carbon-carbon double bond. Alkenyl groups include, without limitation, vinyl, 1-propenyl, 1-butenyl, 1-pentenyl, and 1-hexenyl.

For purposes of the present invention, the term “alkynyl” will be used when the carbon atom attaching the aliphatic group to the rest of the molecule forms part of a carbon-carbon triple bond. Alkynyl groups include, without limitation, ethynyl, 1-propynyl, 1-butynyl, 1-pentynyl, and 1-hexynyl.

The term “cycloaliphatic”, used alone or as part of a larger moiety, refers to a saturated or partially unsaturated cyclic aliphatic ring system having from 3 to about 14 members, wherein the aliphatic ring system is optionally substituted. In some embodiments, the cycloaliphatic is a monocyclic hydrocarbon having 3-8 or 3-6 ring carbon atoms. Nonlimiting examples include cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloaliphatic is a bridged or fused bicyclic hydrocarbon having 6-12, 6-10, or 6-8 ring carbon atoms, wherein any individual ring in the bicyclic ring system has 3-8 members.

In some embodiments, two adjacent substituents on the cycloaliphatic ring, taken together with the intervening ring atoms, form an optionally substituted fused 5- to 6-membered aromatic or 3- to 8-membered non-aromatic ring having 0-3 ring heteroatoms selected from the group consisting of O, N, and S. Thus, the term “cycloaliphatic” includes aliphatic rings that are fused to one or more aryl, heteroaryl, or heterocyclyl rings. Nonlimiting examples include indanyl, 5,6,7,8-tetrahydroquinoxalinyl, decahydronaphthyl, or tetrahydronaphthyl, where the radical or point of attachment is on the aliphatic ring.

The terms “aryl” and “ar-”, used alone or as part of a larger moiety, e.g., “aralkyl”, “aralkoxy”, or “aryloxyalkyl”, refer to a C₆ to C₁₄ aromatic hydrocarbon, comprising one to three rings, each of which is optionally substituted. Preferably, the aryl group is a C₆₋₁₀ aryl group. Aryl groups include, without limitation, phenyl, naphthyl, and anthracenyl. In some embodiments, two adjacent substituents on the aryl ring, taken together with the intervening ring atoms, form an optionally substituted fused 5- to 6-membered aromatic or 4- to 8-membered non-aromatic ring having 0-3 ring heteroatoms selected from the group consisting of O, N, and S. Thus, the term “aryl”, as used herein, includes groups in which an aryl ring is fused to one or more heteroaryl, cycloaliphatic, or heterocyclyl rings, where the radical or point of attachment is on the aromatic ring. Nonlimiting examples of such fused ring systems include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, tetrahydroquinolinyl, tetrahydroisoquinolinyl, fluorenyl, indanyl, phenanthridinyl, tetrahydronaphthyl, indolinyl, phenoxazinyl, benzodioxanyl, and benzodioxolyl. An aryl group may be mono-, bi-, tri-, or polycyclic, preferably mono-, bi-, or tricyclic, more preferably mono- or bicyclic. The term “aryl” may be used interchangeably with the terms “aryl group”, “aryl moiety”, and “aryl ring”.

An “aralkyl” or “arylalkyl” group comprises an aryl group covalently attached to an alkyl group, either of which independently is optionally substituted. Preferably, the aralkyl group is C₆₋₁₀ aryl(C₁₋₆)alkyl, C₆₋₁₀ aryl(C₁₋₄)alkyl, or C₆₋₁₀ aryl(C₁₋₃)alkyl, including, without limitation, benzyl, phenethyl, and naphthylmethyl.

The terms “heteroaryl” and “heteroar-”, used alone or as part of a larger moiety, e.g., heteroaralkyl, or “heteroaralkoxy”, refer to groups having 5 to 14 ring atoms, preferably 5, 6, 9, or 10 ring atoms; having 6, 10, or 14 π electrons shared in a cyclic array; and having, in addition to carbon atoms, from one to four heteroatoms. The term “heteroatom” refers to nitrogen, oxygen, or sulfur, and includes any oxidized form of nitrogen or sulfur, and any quaternized form of a basic nitrogen. Thus, when used in reference to a ring atom of a heteroaryl, the term “nitrogen” includes an oxidized nitrogen (as in pyridine N-oxide). Certain nitrogen atoms of 5-membered heteroaryl groups also are substitutable, as further defined below. Heteroaryl groups include, without limitation, radicals derived from thiophene, furan, pyrrole, imidazole, pyrazole, triazole, tetrazole, oxazole, isoxazole, oxadiazole, thiazole, isothiazole, thiadiazole, pyridine, pyridazine, pyrimidine, pyrazine, indolizine, naphthyridine, pteridine, pyrrolopyridine, imidazopyridine, oxazolopyridine, thiazolopyridine, triazolopyridine, pyrrolopyrimidine, purine, and triazolopyrimidine. As used herein, the phrase “radical derived from” means a monovalent radical produced by removal of a hydrogen radical from the parent heteroaromatic ring system. The radical (i.e., the point of attachment of the heteroaryl to the rest of the molecule) may be created at any substitutable position on any ring of the parent heteroaryl ring system.

In some embodiments, two adjacent substituents on the heteroaryl, taken together with the intervening ring atoms, form an optionally substituted fused 5- to 6-membered aromatic or 4- to 8-membered non-aromatic ring having 0-3 ring heteroatoms selected from the group consisting of O, N, and S. Thus, the terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono-, bi-, tri-, or polycyclic, preferably mono-, bi-, or tricyclic, more preferably mono- or bicyclic. The term “heteroaryl” may be used interchangeably with the terms “heteroaryl ring”, or “heteroaryl group”, any of which terms include rings that are optionally substituted. The term “heteroaralkyl” refers to an alkyl group substituted by a heteroaryl, wherein the alkyl and heteroaryl portions independently are optionally substituted.

As used herein, the terms “aromatic ring” and “aromatic ring system” refer to an optionally substituted mono-, bi-, or tricyclic group having 0-6, preferably 0-4 ring heteroatoms, and having 6, 10, or 14 π electrons shared in a cyclic array. Thus, the terms “aromatic ring” and “aromatic ring system” encompass both aryl and heteroaryl groups.

As used herein, the terms “heterocycle”, “heterocyclyl”, “heterocyclic radical”, and “heterocyclic ring” are used interchangeably and refer to a stable 3- to 7-membered monocyclic, or to a fused 7- to 10-membered or bridged 6- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a heterocyclyl ring having 1-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure, and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquirtolirtyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl.

In some embodiments, two adjacent substituents on a heterocyclic ring, taken together with the intervening ring atoms, form an optionally substituted fused 5- to 6-membered aromatic or 3- to 8-membered non-aromatic ring having 0-3 ring heteroatoms selected from the group consisting of O, N, and S. Thus, the terms “heterocycle”, “heterocyclyl”, “heterocyclyl ring”, “heterocyclic group”, “heterocyclic moiety”, and “heterocyclic radical”, are used interchangeably herein, and include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond between ring atoms. The term “partially unsaturated” is intended to encompass rings having multiple sites of unsaturation, but is not intended to include aryl or heteroaryl moieties, as herein defined.

The terms “haloaliphatic”, “haloalkyl”, “haloalkenyl” and “haloalkoxy” refer to an aliphatic, alkyl, alkenyl or alkoxy group, as the case may be, which is substituted with one or more halogen atoms. As used herein, the term “halogen” or “halo” means F, Cl, Br, or I. The term “fluoroaliphatic” refers to a haloaliphatic wherein the halogen is fluoro, including perfluorinated aliphatic groups. Examples of fluoroaliphatic groups include, without limitation, fluoromethyl, difluoromethyl, trifluoromethyl, 2-fluoroethyl, 2,2,2-trifluoroethyl, 1,1,2-trifluoroethyl, 1,2,2-trifluoroethyl, and pentafluoroethyl.

The term “alkylene” refers to a bivalent alkyl group. An “alkylene chain” is a polymethylene group, i.e., —(CH₂)n—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

An alkylene chain also can be optionally interrupted by a functional group. An alkylene chain is “interrupted” by a functional group when an internal methylene unit is replaced with the functional group. Examples of suitable “interrupting functional groups” include —C(R*)═C(R*)—, —O—, —S—, —S(O)—, —S(O)₂—, —S(O)₂N(R⁺)—, —N(R*)—, —N(R⁺)CO—, —N(R⁺)C(O)N(R⁺)—, —N(R⁺)C(═NR⁺)—N(R⁺)—, —N(R⁺)—C(═NR⁺)—, —N(R⁺)CO₂—, —N(R⁴)SO₂—, —N(R⁺)SO₂N(R⁺)—, —OC(O)—, —OC(O)O—, —OC(O)N(R⁺)—, —C(O)—, —CO₂—, —C(O)N(R⁺)—, —C(O)—C(O)—, —C(═NR⁺)—N(R⁺)—, —C(NR⁺)═N—, —C(═NR⁺)—O—, —C(OR*)═N—, —C(R^{o})═N—O—, or —N(R⁺)—N(R⁺)—. Each R⁺, independently, is hydrogen or an optionally substituted aliphatic, aryl, heteroaryl, or heterocyclyl group, or two R⁺ on the same nitrogen atom, taken together with the nitrogen atom, form a 5-8 membered aromatic or non-aromatic ring having, in addition to the nitrogen atom, 0-2 ring heteroatoms selected from N, O, and S. Each R* independently is hydrogen or an optionally substituted aliphatic, aryl, heteroaryl, or heterocyclyl group.

Examples of C₃₋₆ alkylene chains that have been “interrupted” with —O— include CH₂OCH₂—, —CH₂O(CH₂)₂—, —CH₂O(CH₂)₃—, —CH₂O(CH₂)₄—, —(CH₂)₂OCH₂, —(CH₂)₂O(CH₂)₂—, —(CH₂)₂O(CH₂)₃—, —(CH₂)₃O(CH₂)—, —(CH₂)₃O(CH₂)₂—, and —(CH₂)₄O(CH₂)—. Other examples of alkylene chains that are “interrupted” with functional groups include —CH₂ZCH₂—, —CH₂Z(CH₂)₂—, —CH₂Z(CH₂)₃—, —CH₂Z(CH₂)₄—, —(CH₂)₂ZCH₂—, —(CH₂)₂Z(CH₂)₂—, —(CH₂)₂Z(CH₂)₃—, —(CH₂)₃Z(CH₂)—, —(CH₂)₃Z(CH₂)₂—, and —(CH₂)₄Z(CH₂)—, wherein Z is one of the “interrupting” functional groups listed above.

For purposes of clarity, all bivalent groups described herein are intended to be read from left to right, with a corresponding left-to-right reading of the formula or structure in which the variable appears.

One of ordinary skill in the art will recognize that when an alkylene chain having an interruption is attached to a functional group, certain combinations would not be sufficiently stable for pharmaceutical use. Only stable or chemically feasible compounds are within the scope of the present invention. A stable or chemically feasible compound is one in which the chemical structure is not substantially altered when kept at a temperature from about −80° C. to about +40° C., preferably −20° C. to about +40° C., in the absence of moisture or other chemically reactive conditions, for at least a week, or a compound which maintains its integrity long enough to be useful for therapeutic or prophylactic administration to a patient.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more substituents”, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different.

As used herein, the term “independently selected” means that the same or different values may be selected for multiple instances of a given variable in a single compound.

An aryl (including the aryl moiety in aralkyl, aralkoxy, aryloxyalkyl and the like) or heteroaryl (including the heteroaryl moiety in heteroaralkyl and heteroaralkoxy and the like) group may contain one or more substituents. Examples of suitable substituents on the unsaturated carbon atom of an aryl or heteroaryl group include -halo, —NO₂, —CN, —R*, —C(R*)═C(R*)₂, —C≡C—R*, —OR*, —SR^{o}, —S(O)R^{o}, —SO₂R^{o}, —SO₃R*, —SO₂N(R⁺)₂, —N(R⁺)₂, —NR⁺C(O)R*, —NR⁺C(O)N(R⁺)₂, —N(R⁺)C(═NR⁺)—N(R⁺)₂, —N(R⁺)C(═NR⁺)—R^{o}, —NR⁺CO₂R^{o}, —NR⁺SO₂R^{o}, —NR⁺SO₂N(R⁺)₂, —O—C(O)R*, —O—CO₂R*, —OC(O)N(R⁺)₂, —C(O)R*, —CO₂R*, —C(O)—C(O)R*, —C(O)N(R⁺)₂, —C(O)N(R⁺)—OR*, —C(O)N(R⁺)C(═NR⁺)—N(R⁺)₂, —N(R^{f})C(═NR⁺)—N(R⁺)—C(O)R*, —C(═NR⁺)—N(R⁺)₂, —C(═NR⁺)—OR*, —N(R⁺)—N(R⁺)₂, —C(═NR⁺)—N(R⁺)—OR*, —C(R^{o})═N—OR*, —P(O)(R*)₂, —P(O)(OR*)₂, —O—P(O)—OR*, and —P(O)(NR⁺)—N(R⁺)₂, wherein R^{o} is an optionally substituted aliphatic, aryl, or heteroaryl group, and R⁺ and R* are as defined above, or two adjacent substituents, taken together with their intervening atoms, form a 5-6 membered unsaturated or partially unsaturated ring having 0-3 ring atoms selected from the group consisting of N, O, and S.

An aliphatic group or a non-aromatic heterocyclic ring may be substituted with one or more substituents. Examples of suitable substituents on the saturated carbon of an aliphatic group or of a non-aromatic heterocyclic ring include, without limitation, those listed above for the unsaturated carbon of an aryl or heteroaryl group and the following: ═O, ═S, ═C(R*)₂, ═N—N(R*)₂, ═N—NHC(O)R*, ═N—NHCO₂R^{o}, ═N—NHSO₂R^{o}, or ═N—R*, where each R* and R^{o} is as defined above.

Suitable substituents on a substitutable nitrogen atom of a heteroaryl or non-aromatic heterocyclic ring include —R*, —N(R*)₂, —C(O)R*, —C(O)N(R*)₂, —CO₂R*, —C(O)—C(O)R* —C(O)CH₂C(O)R*, —SO₂R*, —SO₂N(R*)₂, —C(═S)N(R*)₂, —C(═NH)—N(R*)₂, and —NR*SO₂R*; wherein each R* is as defined above. A ring nitrogen atom of a heteroaryl or non-aromatic heterocyclic ring also may be oxidized to form the corresponding N-hydroxy or N-oxide compound. A nonlimiting example of such a heteroaryl having an oxidized ring nitrogen atom is N-oxidopyridyl. A ring sulfur atom of a heterocyclic ring may be oxidized to form the corresponding sulfoxide or sulfone.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, the term “comprises” means “includes, but is not limited to.”

It will be apparent to one skilled in the art that certain compounds of this invention may exist in tautomeric forms, all such tautomeric forms of the compounds being within the scope of the invention. Unless otherwise stated, structures depicted herein are also meant to include all geometric (or conformational) isomers, i.e., (Z) and (E) double bond isomers and (Z) and (E) conformational isomers, as well as all stereochemical forms of the structure; i.e., the R and S configurations for each asymmetric center. Therefore, single stereochemical isomers as well as enantiomeric and diastereomeric mixtures of the present compounds are within the scope of the invention. When a mixture is enriched in one stereoisomer relative to another stereoisomer, the mixture may contain, for example, an enantiomeric excess of at least 50%, 75%, 90%, 99%, or 99.5%.

Unless otherwise stated, structures depicted herein are also meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

In order that this invention be more fully understood, the following preparative examples are set forth. These examples illustrate how to make specific compounds, and are not to be construed as limiting the scope of the invention in any way.

### EXAMPLES

### Abbreviations

AcOH acetic acid
ACN acetonitrile
ATP adenosine triphosphate
BCA bicinchoninic acid
BSA bovine serum albumin
BOC tert-butoxycarbonyl
DABCO 1,4-diazabicyclo[2.22]octane
DCM dichloromethane
DIPEA diisopropyl ethyl amine
DMA dimethylacetamide
DMAP 4-dimethylaminopyridine
DMEM Dulbecco's Modified Eagle's medium
DMF dimethylformamide
DTT dithiothreitol
EDCI 1-(3-dimethylaminopropyl)-3-ethylcarbodiimide
EDTA ethylenediaminetetraacetic acid
EtOAc ethyl acetate
Et₂O ethyl ether
FA formic acid
FBS fetal bovine serum
h hours
HATU O-(7-azabenzotriazol-1-yl)-N,N,N′,N′-tetramethyluronium hexafluorophosphate
MeOH methanol
min minutes
MTT methylthiazoletetrazolium
MWI microwave irradiation
NMP 1-methyl-2-pyrrolidinone
PBS phosphate buffered saline
pTSA p-toluenesulfonic acid
PKA cAMP-dependent protein kinase
sec seconds
rt room temperature
TBTU O-(1H-benzotriazol-1-yl)-N,N,N′,N′-tetramethyluronium tetrafluoroborate
TEA triethylamine
THF tetrahydrofuran
TMB 3,3′,5,5′-tetramethylbenzidine
WST (4-[3-(4-iodophenyl)-2-(4-nitrophenyl)-2H-5-tetrazolio]-1,3-benzene disulfonate sodium salt)
m/z mass to charge
MS mass spectrum
LC/MS liquid chromatography mass spectrum
HRMS high resolution mass spectrum

### Analytical LC-MS Methods

Spectra were run on a Phenominex Luna 5 μm C18 50×4.6 mm column on a Hewlett-Packard HP1100 at 2.5 ml/min for a 3 minute run using the following gradients:
Formic Acid (FA): Acetonitrile containing zero to 100 percent 0.1% formic acid in water.
Ammonium Acetate (AA): Acetonitrile containing zero to 100 percent 10 mM ammonium acetate in water.

### Example 1

### 5-fluoro-3,4-dihydro-1,8-naphthyridin-2(1H)-one

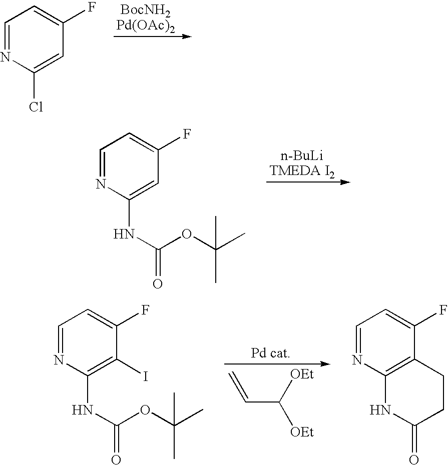

### Step 1 Preparation of tert-butyl (4-fluoropyridin-2-yl)carbamate

Palladium acetate (341 mg, 1.52 mmol) and 4,5-bis(diphenylphosphino)-9,9-dimethylxanthene (1.76 g, 3.04 mmol) were added to a 3-necked round bottomed flask, and the flask was purged three times with argon. Degassed 1,4-dioxane (240 mL) was added and the mixture was stirred and degassed again with argon. To this solution was added a solution of 2-chloro-4-fluoropyridine (20 g, 152 mmol) in degassed 1,4-dioxane (120 mL), t-butyl carbamate (19.6 g, 167 mmol), NaOH (8.88 g, 222 mmol) and degassed water (4.0 mL, 222 mmol). The resulting mixture was stirred at 100° C. After 1.5 h, the reaction mixture was cooled to rt and filtered through a pad of Celite. The pad was washed well with dioxane and the filtrate was concentrated under reduced pressure to dryness. The resulting solid was recrystallized from 2-propanol (˜250 mL) to give 25.65 g of a pale yellow crystalline solid (79.5% yield). LC/MS: (FA) ES+ 213. ¹H NMR (400 MHz, d6 DMSO): δ 10.14 (s, 1H), 8.28-8.25 (m, 1H), 7.62-7.58 (m, 1H), 6.97-6.93 (m, 1H), and 1.47 (s, 9H).

### Step 2 Preparation of tert-butyl (4-fluoro-3-iodopyridin-2-yl)carbamate (method 1)

An oven-dried 3-neck round bottom flask equipped with an overhead stirrer, temperature probe, and addition funnel was charged with tert-butyl (4-fluoropyridin-2-yl)carbamate (31.8 g, 150 mmol), TMEDA (56.6 mL, 375 mmol) and THF (200 mL). The solution was cooled to −78° C. and a solution of n-BuLi (2.50 M in hexane, 150 mL, 375 mmol) was added dropwise so that the reaction mixture temperature remained below −70° C. The reaction mixture was stirred at −78° C. for 1 h, and a solution of I₂ (95.2 g, 375 mmol) in THF (160 mL) was added via addition funnel. The addition was controlled to keep the reaction mixture temperature below −70° C., and the resulting mixture was stirred at −78° C. for 1 h. A solution of NaHSO₄ (61 g, 580 mmol) in water (200 mL) was added to the reaction mixture as it warmed to rt. Ethyl acetate was added and the 2 phase mixture was stirred at rt for 1 h. Water (500 mL) was added and the phases were separated. The aqueous phase was extracted with EtOAc (3×400 mL), the organic phases were combined, dried over MgSO₄, filtered and concentrated to give an off white solid. This solid was suspended in DCM (50 mL) and the solid was isolated by filtration and washed with a minimum of DCM. The filtrate was concentrated and filtered to give a second crop of product. The solids were combined and dried under vacuum to give 45.63 g of a white solid (86% yield). LC/MS: (FA) ES+ 339. ¹H NMR (400 MHz, d6 DMSO): δ 9.47 (s, 1H), 8.33-8.30 (m, 1H), 7.20-7.17 (m, 1H), and 1.44 (s, 9H).

### Preparation of tert-butyl (4-fluoro-3-iodopyridin-2-yl)carbamate (method 2)

To a solution of tert-butyl (4-fluoropyridin-2-yl)carbamate (2.00 g, 9.42 mmol) in THF (40 mL), at 2° C. under an atmosphere of nitrogen, was added 1.0M dibutylmagnesium in hexane (20.7 mL, 20.7 mmol) dropwise, maintaining the internal temperature below 7° C. When addition was complete, the reaction was allowed to warm to rt, and then heated at 50° C. for 3 h. The reaction was then allowed to cool to rt. A solution of iodine (10.5 g, 41.5 mmol) in THF (30 ml) was added dropwise over approximately 30 min., maintaining the internal temperature between 19° C. and 22° C. by the occasional use of an ice bath. The reaction mixture was allowed to stir overnight at rt. A 0.5 M solution of sodium ascorbate in water (60 mL, 300 mmol) was added and the phases were separated. The aqueous phase was extracted with EtOAc (40 mL) and the combined organic phases were washed with brine, dried (MgSO₄) and evaporated to approximately 4 mL. On standing for 1 h. some solids were formed. Heptane (10 mL) and EtOAc (2 mL) were added and the suspension was stirred at rt for 30 min. The precipitate was filtered off, and the solid was washed with heptane and dried under vacuum at 40° C. to provide 2.31 g of yellow solid (72.5% yield).

### Step 3 Preparation of 5-fluoro-3,4-dihydro-1,8-naphthyridin-2(1H)-one

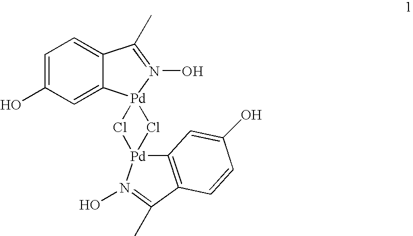

A round bottom flask was charged with tert-butyl (4-fluoro-3-iodopyridin-2-yl)carbamate (20.0 g, 59.2 mmol), 3,3-diethoxy-1-propene (13.5 mL, 88.7 mmol), DMF (150 mL), water (50 mL), DIPEA (15.4 mL, 88.7 mmol) and Pd catalyst 1 (Corma, A.; Garcia, H.; Leyva, A. Tetrahedron 61, 9848, 2005) (480 mg, 0.827 mmol) and the reaction mixture was stirred in an oil bath at 140° C. After 5 h, the reaction mixture was cooled in a refrigerator for 2 days. The precipitate was isolated by filtration, washed with diethyl ether, and dried to give 3.25 g of pink needles. The filtrate was concentrated to give a reddish semi-solid. This material was redissolved in DCM, and the solution was passed through 200 g of SiO₂. Concentration of the resulting solution provided a red/orange residue which was recrystallized from 2-propanol (150 mL) to give 9.4 g of a pink solid. Purification of this pink solid by column chromatography (SiO₂, elution with 0-75% EtOAc/DCM) provided 1.41 g of a white powder. Overall, 4.66 g of final product was isolated (47% yield). LC/MS: (FA) ES+ 167. ¹H NMR (300 MHz, d6 DMSO): δ 10.67 (s, 1H), 8.13-8.08 (m, 1H), 6.93-6.88 (m, 1H), 2.87 (t, 2H), and 2.51 (t, 2H). This reaction may also be carried out under the same conditions using Pd(OAc)₂ as catalyst, both in the presence or absence of tri-o-tolylphosphine as ligand.

In a method analogous to that described for 5-fluoro-3,4-dihydro-1,8-naphthyridin-2(1H)-one, 5-chloro-3,4-dihydro-1,8-naphthyridin-2(1H)-one was prepared from 2,4-dichloropyridine. LC/MS: (AA) ES+ 183. ¹H NMR (400 MHz, d6 DMSO): δ 10.68 (s, 1H), 8.07-8.06 (m, 1H), 7.13-7.11 (m, 1H), 2.96 (t, 2H), and 2.55 (t, 2H).

### Example 2

### (7R)-7-amino-5,6,7,8-tetrahydronaphthalen-2-ol hydrobromide

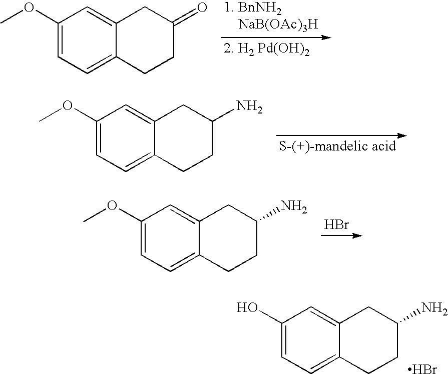

### Step 1 Preparation of N-benzyl-7-methoxy-1,2,3,4-tetrahydronaphthalen-2-amine

7-Methoxy-2-tetralone (18.37 g, 104 mmol) was dissolved in methylene chloride (400 mL), to which was subsequently added benzylamine (11.4 mL, 104 mmol). After stirring for 15 min, sodium triacetoxyborohydride (30.9 g, 146 mmol) and AcOH (5.9 mL, 100 mmol) were added to the dark mixture. The mixture lightened on addition of AcOH and the reaction mixture was allowed to stir overnight (15 h) at rt under nitrogen. The red/brown reaction mixture was diluted with DCM (400 mL) and washed with 1M sodium hydroxide solution (4×200 mL). The washings were combined and extracted with DCM (150 mL). The extracts were combined and washed with brine, dried over sodium sulfate, filtered and concentrated under reduced pressure to afford the benzylamine product (28.0 g, 100% as a brown oil). LC/MS: (AA) ES+ 268.

### Step 2 Preparation of 7-methoxy-1,2,3,4-tetrahydronaphthalen-2-amine hydrochloride

N-benzyl-7-methoxy-1,2,3,4-tetrahydronaphthalen-2-amine (28.0 g, 105 mmol) was dissolved in reagent ethanol (400 mL). AcOH (92 mL, 1600 mmol) was added. The dark solution was degassed under reduced pressure and backfilled with nitrogen. Palladium hydroxide (7.0 g, 20% on carbon) was added. Hydrogen gas was bubbled through the reaction for 5 minutes then the reaction was placed under an atmosphere of hydrogen (balloon) and stirred at rt for 24 h, at which time the reaction was complete. Hydrogen was removed under reduced pressure and the flask was backfilled with nitrogen. The reaction mixture was filtered through a Celite pad, which was subsequently washed thoroughly. The filtrate was concentrated under reduced pressure and further dried in vacuo. The resulting dark oil was dissolved in ether and the solution acidified by the addition of 2.0 M HCl in ether (100 mL) added in portions by pipet. A gummy precipitate formed on acidification. Thorough sonication of the gum provided a white to light tan silty precipitate which was collected by filtration and dried in vacuo. Yield: 22.4 g (92%). LC/MS: (AA) ES+ 178.

### Step 3 Preparation of (2R)-7-methoxy-1,2,3,4-tetrahydronaphthalen-2-amine

7-methoxy-1,2,3,4-tetrahydronaphthalen-2-amine hydrochloride (21.7 g) was partitioned between 1M NaOH (200 mL) and ethyl acetate (200 mL). The aqueous phase was extracted with ethyl acetate (3×200 mL). The extracts were combined, washed with brine, dried over sodium sulfate, filtered and concentrated to afford a brown oil (17.7 g, 99.6 mmol).

To a stirred solution of S-(+)-mandelic acid (15.4 g, 101 mmol), isopropyl alcohol (78 mL) and 80/20 methanol/water (51 mL) was added a solution of the free base of 7-methoxy-1,2,3,4-tetrahydronaphthalen-2-amine in toluene (10 mL) and 80/20 methanol/water (40 mL) via a dropping funnel. After addition was completed, the mixture was stirred at reflux for 30 min. The mixture was then cooled to rt. The mixture was allowed to stand at rt over the weekend. The resulting solids (16.95 g) were isolated by filtration, washed with minimal ethyl acetate and dried in vacuo. The salt was then suspended in an 80/20 methanol/water solution (55 mL) and warmed to reflux. Additional 80/20 methanol/water solution was added until the solution became homogeneous (about 10 mL). Upon complete dissolution, the solution was stirred at reflux 30 min, cooled to room temperature and allowed to stand undisturbed over night. The resulting white solids which precipitated were collected by suction filtration (11.94 g) and dried in vacuo. The solids were recrystallized as above from 80/20 methanol/water (ca. 60 mL) to afford 10.05 g of the S-(+)-mandelate salt ([α]═+90°, c═0.5, MeOH (Eur. J. Med. Chem. 1994, 29, 259-267)). The salt was partitioned between 4.00 M of sodium hydroxide in water (30.0 mL) and ethyl acetate (100 mL). The phases were separated and the aqueous phase was extracted with ethyl acetate (2×100 mL). The extracts were combined, washed with brine (35 mL), dried over sodium sulfate, filtered and concentrated to afford the desired amine (5.39 g, 60% of theoretical) as an oil. LC/MS: (AA) ES+ 178.

### Step 4 Preparation of (7R)-7-amino-5,6,7,8-tetrahydronaphthalen-2-ol hydrobromide

A suspension of (2R)-7-methoxy-1,2,3,4-tetrahydronaphthalen-2-amine (5.92 g, 33.7 mmol) in hydrobromic acid (48% in water, 80 mL) was warmed to reflux. After 1.75 h, the reaction solution was cooled to rt. The solvent was removed under reduced pressure. The oily residue was twice dissolved in ethanol (100 mL) and concentrated to dryness. The resulting oil was further dried in vacuo, affording the desired product as a brown waxy solid (9.128 g, 99% yield, ([α]+91°, c═0.5, MeOH). (Eur. J. Med. Chem. 1994, 29, 259-267). LC/MS: (AA) ES+ 164.

### Example 3

### 5-{[(7R)-7-amino-5,6,7,8-tetrahydronaphthalen-2-yl]oxy}-3,4-dihydro-1,8-naphthyridin-2(1H)-one. Method 1

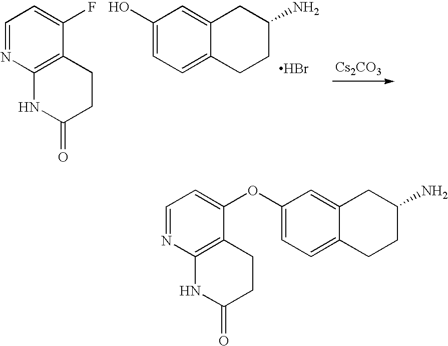

A mixture of (7R)-7-amino-5,6,7,8-tetrahydronaphthalen-2-ol hydrobromide (16.7 g, 68.2 mmol), 5-fluoro-3,4-dihydro-1,8-naphthyridin-2(1H)-one (11.36 g, 64.95 mmol), and cesium carbonate (63.49 g, 194.9 mmol) in DMF (216 mL) was stirred at 140° C. for 2 h. The reaction was not complete, so additional (7R)-7-amino-5,6,7,8-tetrahydronaphthalen-2-ol hydrobromide (1.70 g, 6.82 mmol) was added. After an additional 1 h at 140° C., the reaction mixture was cooled to rt, and carefully treated with a solution of HCl (1 M). The reaction mixture was then diluted with DCM and filtered through Celite. The phases were separated and the aqueous phase was brought to pH 7 by the addition of a solution of NaOH (1.0 M). The brown precipitate was then removed by filtration through Celite, and the filtrate was washed with DCM. The aqueous phase was then brought to pH 14 by addition of a solution of NaOH (1.0 M) and an off-white precipitate formed. This suspension was treated with solid NaCl and stirred for 1 h. The solid was isolated by filtration and dried under vacuum to give a 17.7 g of an off-white solid (88% yield). LC/MS: (AA) ES+ 310. ¹H NMR (400 MHz, d6 DMSO) δ: 10.47 (s, 1H), 7.94 (d, 1H), 7.12 (d, 1H), 6.85-6.81 (m, 2H), 6.26 (d, 1H), 3.03-2.96 (m, 1H), 2.89 (t, 2H), 2.85-2.79 (m, 1H), 2.76-2.66 (m, 1H), 2.51 (t, 2H), 2.44-2.38 (m, 1H), 1.90-1.84 (m, 1H), 1.65 (br s, 2H), and 1.50-1.40 (m, 1H).

### Example 4

### 5-{[(7R)-7-amino-5,6,7,8-tetrahydronaphthalen-2-yl]oxy}-3,4-dihydro-1,8-naphthyridin-2(1H)-one, Method 2

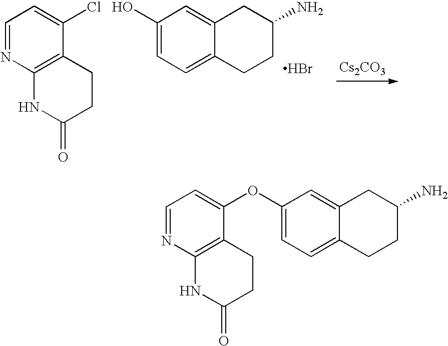

A mixture of (7R)-7-amino-5,6,7,8-tetrahydronaphthalen-2-ol hydrobromide (735 mg, 3.01 mmol), 5-chloro-3,4-dihydro-1,8-naphthyridin-2(1H)-one (500 mg, 2.74 mmol), and cesium carbonate (2.68 g, 8.21 mmol) in DMF (10 mL) was heated in a microwave reactor at 250° C. for 6 min. The reaction mixture was cooled to rt, and the solvents were evaporated. 1N NaOH solution (100 mL) was added and the mixture was extracted with DCM (150 mL×3). The organic layers were dried (Na₂SO₄) and evaporated. Purification by column chromatography (SiO₂, elution with 50:40:9:1 DCM:MeCN:MeOH:NH₄OH) provided 580 mg of an off-white solid (65% yield). LC/MS: (AA) ES+ 310. ¹H NMR (400 MHz, d6 DMSO) δ: 10.47 (s, 1H), 7.94 (d, 1H), 7.12 (d, 1H), 6.85-6.81 (m, 2H), 6.26 (d, 1H), 3.03-2.96 (m, 1H), 2.89 (t, 2H), 2.85-2.79 (m, 1H), 2.76-2.66 (m, 1H), 2.51 (t, 2H), 2.44-2.38 (m, 1H), 1.90-1.84 (m, 1H), 1.65 (br s, 2H), and 1.50-1.40 (m, 1H).

### Example 5

### tert-butyl 3-formyl-5-(trifluoromethyl)benzoate

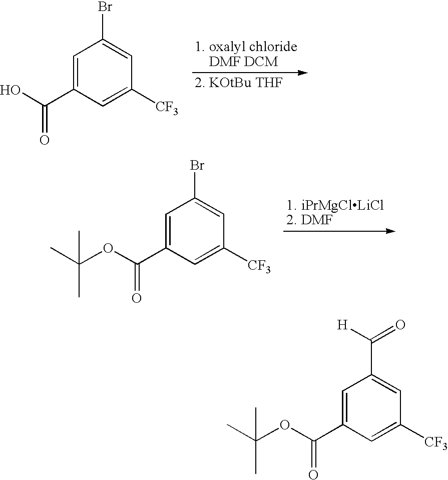

Step 1 Preparation of tert-butyl 3-bromo-5-(trifluoromethyl)benzoate

To a solution of 3-bromo-5-(trifluoromethyl)benzoic acid (95.2 g, 354 mmol) in DCM (470 mL), was added DMF (11.0 mL, 567 mmol). Oxalyl chloride (48.0 mL, 567 mmol) was added dropwise. The reaction was stirred for 18 h. The solvents were evaporated and the residue was azeotroped with toluene (2×). THF (470 mL) was added and the reaction was cooled to 0° C. Potassium tert-butoxide (1.0 M solution in THF, 708 mL, 708 mmol) was added dropwise. When addition was complete the reaction was stirred at it for 1 h. LC/MS indicated that the reaction was complete. Water was added and the mixture was extracted into EtOAc (2×). The combined organic phases were washed with water and brine, dried (Na₂SO₄) and evaporated. The residue was purified by filtration through silica in a 3 L fritted funnel, eluting with 5% EtOAc/hexane to provide the desired product as an oil (105 g, 91.1%). ¹H NMR (300 MHz, CDCl₃): δ 8.28 (s, 1H), 8.16 (s, 1H), 7.91 (s, 1H), and 1.61 (s, 9H).

### Step 2 Preparation of tert-butyl 3-formyl-5-(trifluoromethyl)benzoate

A solution of tert-butyl 3-bromo-5-(trifluoromethyl)benzoate (90.0 g, 277 mmol) in THF (674 mL) was degassed with argon for 15 min, and cooled to −20° C. A 1.3 M solution of isopropylmagnesiumchloride lithium chloride complex in THF (256 mL, 332 mmol) was added dropwise over approximately 30 min, maintaining the temperature during addition between −20° C. and −30° C. When addition was complete the reaction was stirred at −20° C. to −30° C. for 1 h. DMF (23.6 mL, 304 mmol) was added dropwise, maintaining the temperature between −20° C. and −30° C., and then the reaction was stirred at this temperature for a further 30 min. The reaction was then allowed to warm to 0° C. TLC (10% EtOAc/hexane) showed that the reaction was complete. 1.0 M HCl solution (111 mL, 1.11 mmol) was added and the reaction was allowed to stir at rt for 45 min. Et₂O was added and the layers were separated. The aqueous phase was extracted with Et₂O and the combined organic phases were washed with saturated NaHCO₃ solution, then brine, dried (Na₂SO₄) and evaporated. The residue was purified by filtration through silica in a 3 L fritted funnel, eluting with hexane, 10% DCM/hexane 20% DCM/hexane then 10% EtOAc/hexane to provide the desired product as an off-white solid (52.1 g, 68.7%). ¹H NMR (300 MHz, CDCl₃): δ 10.13 (s, 1H), 8.64 (s, 1H), 8.48 (s, 1H), 8.30 (s, 1H), and 1.64 (s, 9H).

### Example 6

### 3-{[(tert-butoxycarbonyl)(methyl)amino]methyl}-5-(trifluoromethyl)benzoic acid

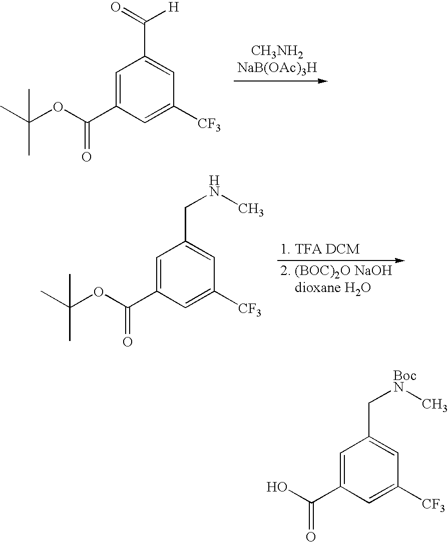

### Step 1 Preparation of tert-butyl 3-[(methylamino)methyl]-5-(trifluoromethyl)benzoate

A solution of 2.0 M of methylamine in tetrahydrofuran (10.0 mL, 20.0 mmol) was added to a solution of tert-butyl 3-formyl-5-(trifluoromethyl)benzoate (2.20 g, 8.02 mmol) in methylene chloride (80 mL). This solution was stirred at rt for 15 minutes, and then sodium triacetoxyborohydride (4.25 g, 20.0 mmol) was added. The resulting solution was stirred at rt overnight. The reaction mixture was diluted with saturated sodium bicarbonate solution (50 mL) and extracted three times with methylene chloride. The organic phase was washed with water and brine, dried over Na₂SO₄, filtered, and concentrated. Purification by column chromatography (SiO₂, elution with 0-10% methanol in ethyl acetate) provided a 1.05 g of a white solid (45% yield). LC/MS: (FA) ES+ 290.

### Step 2 Preparation of 3-[(methylamino)methyl]-5-(trifluoromethyl)benzoic acid.HCl

A solution of tert-butyl 3-[(methylamino)methyl]-5-(trifluoromethyl)benzoate (1.00 g, 3.46 mmol) was dissolved in DCM (6.0 mL) and trifluoroacetic acid (2.66 mL, 3.46 mmol) was added. The resulting solution was stirred at rt for 120 min, and then the solvents were removed in vacuo. The residue was dissolved in methylene chloride (1.0 mL) and 2.0 M of hydrochloric acid in ether (5.0 mL, 10.0 mmol) was added. The solvent was evaporated to dryness and gave 895 mg of a white solid (96% yield). LC/MS: (AA) ES+ 234.

### Step 3 Preparation of 3-{[(tert-butoxycarbonyl)(methyl)amino]methyl}-5-(trifluoromethyl)benzoic acid

3-[(methylamino)methyl]-5-(trifluoromethyl)benzoic acid.HCl (3, 0.895 g, 3.32 mmol) was added 1,4-dioxane (10 mL), water (7.0 mL) and 1.0 M of sodium hydroxide (13.0 mL, 13.0 mmol). Then di-tert-butyldicarbonate (1.45 g, 6.64 mmol) was added and the resulting solution was stirred at rt for 90 min. Dioxane was removed under reduced pressure and pH was adjusted to 3 by the addition of 1N HCl. The aqueous phase was extracted three times with ethyl acetate. The organic phase was washed with brine, dried over Na₂SO₄, filtered, and concentrated. Purification by column chromatography (SiO₂, elution with 0-15% methanol in methylene chloride) provided a 1.01 g of a white solid (91% yield). LC/MS: (FA) ES− 332

Compounds in the following table were prepared from the appropriate starting materials in a method analogous to that described for 3-([(tert-butoxycarbonyl)(methyl)amino]-methyl)-5-(trifluoromethyl)benzoic acid and the corresponding intermediates:

| | |
|---|---|
| 3-((tert-butoxycarbonyl(2,2-difluoroethyl)amino)methyl)- | LC/MS: (FA) |
| 5-(trifluoromethyl)benzoic acid | ES− 382. |
| 3-{[3-(hydroxymethyl)morpholin-4-yl]methyl}-5- | LC/MS: (AA) |
| (trifluoromethyl)benzoic acid | ES+ 320. |
| 3-{[(tert-butoxycarbonyl)(ethyl)amino]methyl}-5- | LC/MS: (FA) |
| (trifluoromethyl)benzoic acid | ES⁻ 346. |

### Example 7

### 3-[(dimethylamino)methyl]-5-(trifluoromethyl)benzoic acid.Li salt

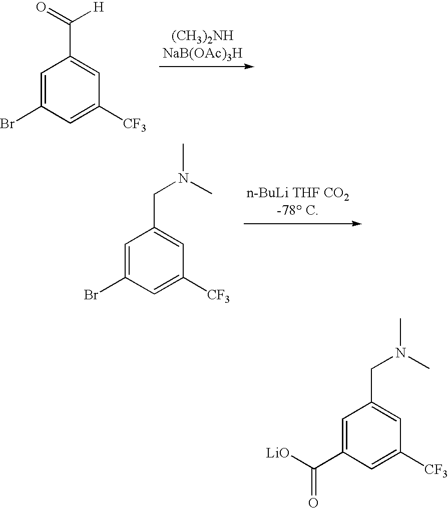

### Step 1 Preparation of 1-[3-bromo-5-(trifluoromethyl)phenyl]-N,N-dimethylmethanamine

To a solution of 3-bromo-5-(trifluoromethyl)benzaldehyde (30.0 g, 118.6 mmol) in methylene chloride (150 mL) was added a solution of 2.0 M of dimethylamine in THF (118 mL) and the reaction was stirred at rt for 15 min. The reaction was cooled to 0° C. and sodium triacetoxyborohydride (37.7 g, 178 mmol) was added. The resulting mixture was warmed to rt and stirred for 3 hours. The solvents were evaporated; saturated sodium bicarbonate solution was added and the resulting mixture was extracted three times with ethyl acetate. The organic phase was washed with brine, dried over Na₂SO₄, filtered, and concentrated. Purification by column chromatography (SiO₂, elution with 10-40% ethyl acetate in hexanes) provided a 24.9 g of a colorless oil (74% yield). LC/MS: (FA) ES+ 282.

### Step 2 Preparation of 3-[(dimethylamino)methyl]-5-(trifluoromethyl)benzoic acid.Li salt

To a solution of 1-[3-bromo-5-(trifluoromethyl)phenyl]-N,N-dimethylmethanamine (2.0 g, 7.1 mmol) in THF (40 mL) at −78° C. was added dropwise a solution of 2.50 M of n-butyllithium in hexane (3.12 mL, 7.81 mmol). The resulting mixture was stirred at −78° C. for 20 min. Crushed solid CO₂ was added and the mixture was stirred at −78° C. for another 15 min. The reaction was quenched by the addition of water (0.156 mL), and allowed to warm to room temperature. The solvents were evaporated and the solid was dried overnight under vacuum to give a 1.38 g of a white solid (77%). LC/MS: (FA) ES+ 248.

### Example 8

### 3-(aminomethyl)-5-(trifluoromethyl)benzoic acid

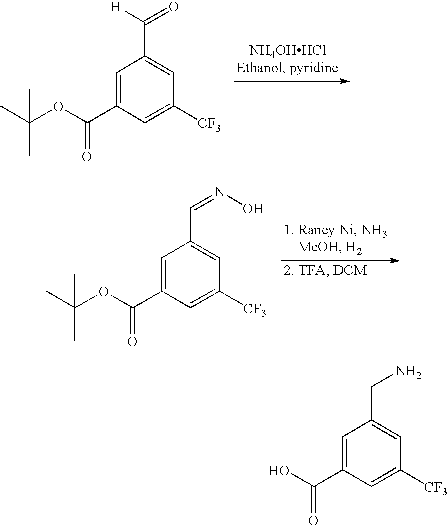

### Step 1 Preparation of tert-butyl 3-[(Z)-(hydroxyimino)methyl]-5-(trifluoromethyl)benzoate

To a solution of tert-butyl 3-formyl-5-(trifluoromethyl)benzoate (1.75 g, 6.40 mmol), in EtOH (60 mL) was added pyridine (2.58 mL, 31.9 mmol) and hydroxylamine hydrochloride (887 mg, 12.8 mmol). The reaction mixture was allowed to stir at rt for 5 h. After this time LC/MS showed complete reaction. Water was added and the mixture was extracted into EtOAc. The organic phase was washed with brine, dried (Na₂SO₄) and evaporated to provide the desired product as a yellow oil (1.97 g, quant). LC/MS: (FA) ES+ 290.

### Step 2 Preparation of tent-butyl 3-(aminomethyl)-5-(trifluoromethyl)benzoate

To a solution of tert-butyl 3-[(Z)-(hydroxyimino)methyl]-5-(trifluoromethyl)benzoate (1.97 g, 6.81 mmol) in 7.0 M ammonia in methanol (50.0 mL) was added Raney Nickel (2800 slurry in water 50% by volume, 0.50 mL). The mixture was treated with hydrogen gas at 50 psi for 18 h. The mixture was then diluted with MeOH and filtered through celite. The filtrate was evaporated and the residue was dissolved in ethyl acetate, washed with brine, dried (Na₂SO₄) and evaporated to provide the desired compound as a yellow oil (1.24 g, 66.1%). LC/MS: (FA) ES+ 276.

### Step 3 Preparation of 3-(aminomethyl)-5-(trifluoromethyl)benzoic acid

To a solution of tert-butyl 3-(aminomethyl)-5-(trifluoromethyl)benzoate (1.24 g, 4.50 mmol) in DCM (40.0 mL) at 0° C., was added trifluoroacetic acid (3.47 mL, 45.1 mmol) dropwise. The reaction was allowed to warm to rt and was stirred overnight. The solvents were evaporated and the residue was triturated with Et₂O to provide the desired compound as a white solid (892 mg, 90.4%). LC/MS: (FA) ES+ 220.

### Example 9

### 3-{1-[(tert-butoxycarbonyl)amino]-1-methylethyl}-5-(trifluoromethyl)benzoic acid (Method 1)

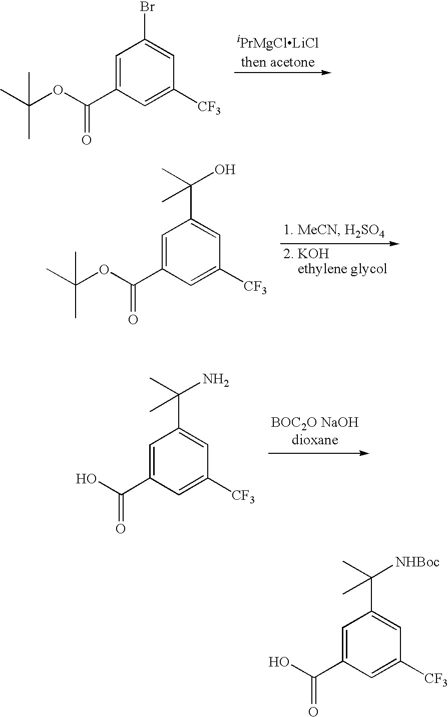

### Step 1 Preparation of tert-butyl 3-(1-hydroxy-1-methylethyl)-5-(trifluoromethyl)benzoate

A solution of tert-butyl 3-bromo-5-(trifluoromethyl)benzoate (35.0 g, 108 mmol) in THF (262 mL) was degassed with argon and cooled to −20° C. A 1.3 M solution of isopropylmagnesium chloride lithium chloride complex in THF (99.4 mL, 129 mmol) was added dropwise, maintaining the temperature of the reaction at −20 to −30° C. When addition was complete, the reaction was stirred at this temperature for 90 min. Acetone (8.69 mL, 118 mmol) was added dropwise maintaining the temperature of the reaction at −20 to −30° C., then the reaction was stirred at this temperature for 30 min. The reaction was allowed to warm to 0° C. and stirred for 45 min. A solution of 1 M HCl (430 mL, 430 mmol) was added, and the reaction was warmed to rt and stirred for 45 min. Et₂O was added and the phases were separated. The aqueous phase was extracted with further Et₂O and the combined organic phases were washed with sat NaHCO₃ solution, then brine, dried (Na₂SO₄) and evaporated. The residue was purified by chromatography on silica (elution with 5% to 20% EtOAc/hexane) to provide the desired compound as a pale yellow oil (16.2 g, 49.3%) LC/MS: (FA) ES+ 287 (M-18)

### Step 2 Preparation of 3-[1-(acetylamino)-1-methylethyl]-5-(trifluoromethyl)benzoic acid

To a solution of tert-butyl 3-(1-hydroxy-1-methylethyl)-5-(trifluoromethyl)benzoate (5.13 g, 16.8 mmol) in acetonitrile (160 mL), was added concentrated sulfuric acid (3.59 mL, 67.4 mmol) dropwise. The reaction was stirred at rt for 3 h. EtOAc was added and the mixture was washed with water (2×). The organic phase was extracted with 1 N NaOH solution (3×) and the basic aqueous phases were acidified to pH 1 by the addition of 6 N HCl solution. The aqueous phase was then extracted into EtOAc (2×) and the organic phases were dried (Na₂SO₄), filtered, and evaporated to provide the desired compound as a white solid (4.48 g, 91.9%). LC/MS: (FA) ES+ 290

### Step 3 Preparation of 3-(1-amino-1-methylethyl)-5-(trifluoromethyl)benzoic acid

A mixture of 3-[1-(acetylamino)-1-methylethyl]-5-(trifluoromethyl)benzoic acid (4.48 g, 15.5 mmol), 1,2-ethanediol (20 mL) and KOH (8.69 g, 155 mmol) was heated at 150° C. for 3 days. The reaction was cooled to rt and approximately 20 mL of water as added. The mixture was washed with Et₂O (2×) and the aqueous phase was acidified to pH 5 by the addition of 6 N HCl solution. The precipitate was filtered off and washed with water. The solid was collected, triturated with water and dried under vacuum to provide the desired compound as a white solid (3.84 g, 100%). LC/MS: (FA) ES+ 248.

### Step 4 Preparation of 3-{1-[(tert-butoxycarbonyl)amino]-1-methylethyl}-5-(trifluoromethyl)benzoic acid

To a solution of 3-(1-amino-1-methylethyl)-5-(trifluoromethyl)benzoic acid (17.2 g, 69.5 mmol) in dioxane (180 mL), was added a solution of 1 N NaOH (208 mL, 208 mmol) and di-tert-butyldicarbonate (60.7 g, 278 mmol). The reaction mixture was stirred overnight. The dioxane was evaporated and the residue was diluted with water and washed with Et₂O (2×). EtOAc was added to the aqueous phase followed by addition of 6 N HCl to acidify the aqueous phase to pH 1. The mixture was extracted with EtOAc (2×). The organic phases were washed with water then brine, dried (Na₂SO₄) and evaporated. The residue was purified by filtration through silica (elution with DCM, then 10% MeOH/1% AcOH/DCM) followed by trituration of the product with DCM. The product was dried under vacuum to provide the desired compound as a white solid (15.0 g, 62.2%). LC/MS: (FA) ES− 346.

Compounds in the following table were prepared from the appropriate starting materials in a method analogous to that described for 3-{1-[(tert-butoxycarbonyl)amino]-1-methylethyl}-5-(trifluoromethyl)benzoic acid and the corresponding intermediates:

| | |
|---|---|
| 3-{1-[(tert-butoxycarbonyl)amino]cyclobutyl}-5- | LC/MS: (FA) |
| (trifluoromethyl)benzoic acid | ES+ 358. |
| 3-[1-(diphenylmethyl)-3-hydroxyazetidin-3-yl]-5- | LC/MS: (AA) |
| (trifluoromethyl)benzoic acid | ES+ 428. |

### Example 10

### 3-{(1S)-1-[(tert-butoxycarbonyl)amino]-2-hydroxyethyl}-5-(trifluoromethyl)benzoic acid

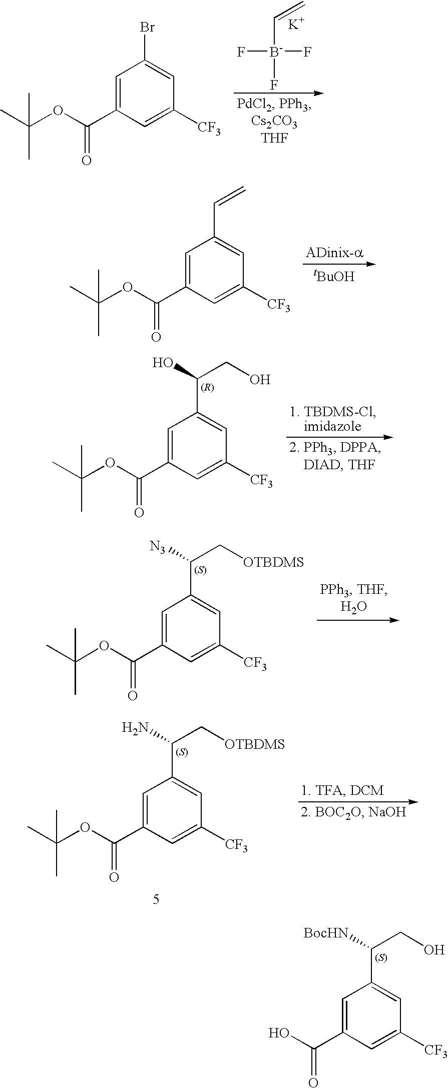

### Step 1 Preparation of tert-butyl 3-(trifluoromethyl)-5-vinylbenzoate

A mixture of tert-butyl 3-bromo-5-(trifluoromethyl)benzoate (1.83 g, 5.60 mmol), potassiumvinyltrifluoroborate (867 mg, 6.50 mmol), triphenylphosphine (88.6 mg, 0.34 mmol), palladium (II) chloride (20.0 mg, 0.11 mmol), and cesium carbonate (5.50 g, 16.9 mmol) in THF (13.7 mL) and water (1.52 mL) was heated in a sealed tube at 85° C., overnight. The reaction mixture was cooled to rt, and diluted with water (20.3 mL). The mixture was extracted with DCM (2×), and the organic phases were dried (MgSO₄) and evaporated. The residue was purified by chromatography on silica (elution with hexane) to provide the desired product as a yellow liquid which was contaminated with approximately 10% bromide starting material (1.0 g, 65.2%). ¹H NMR (300 MHz, CDCl₃): δ 8.18 (s, 1H), 8.10 (s, 1H), 7.78 (s, 1H), 6.77 (dd, 1H), 5.89 (d, 1H), 5.43 (d, 1H), and 1.62 (s, 9H).

### Step 2 Preparation of tert-butyl 3-[(1R)-1,2-dihydroxyethyl]-5-(trifluoromethyl)benzoate

A solution of AD-mix-alpha (2.25 g), in tert-butyl alcohol (8.17 mL) and water (8.17 mL) was cooled in an ice bath, and tert-butyl 3-(trifluoromethyl)-5-vinylbenzoate (440 mg, 1.60 mmol) dissolved in a minimum amount of tert-butyl alcohol was added. The reaction mixture was allowed to stir overnight at rt. Sodium sulfite (2.25 g, 17.8 mmol) was added and the mixture was stirred for 30 min. The solvents were evaporated and water was added. The mixture was extracted into EtOAc (2×), and the organic phases were dried and evaporated. The residue was purified by chromatography on silica (elution with 0% to 20% EtOAc/DCM) to provide the desired compound (390 mg, 78.8%, 92.8% ee). ¹H NMR (300 MHz, CDCl₃): δ 8.14 (s, 2H), 7.83 (s, 1H) 4.94 (dd, 1H), 3.84 (dd, 1H), 3.66 (dd, 1H), and 1.60 (s, 9H).

### Step 3 Preparation of tert-butyl 3-((1R)-2-{[tert-butyl(dimethyl)silyl]oxy}-1-hydroxyethyl)-5-(trifluoromethyl)benzoate

To a solution of tert-butyl 3-[(1R)-1,2-dihydroxyethyl]-5-(trifluoromethyl)benzoate (194 mg, 0.63 mmol) in DMF (6 mL) at 0° C. was added tert-butyldimethylsilyl chloride (114 mg, 0.76 mmol) and imidazole (64.7 mg, 0.95 mmol). The reaction mixture was allowed to warm to room temperature and stirred for 4 h. LC/MS showed complete reaction. Water was added and the mixture was extracted into EtOAc. The organic phase was washed with water (3×), then brine, dried (Na₂SO₄) and evaporated. The residue was purified by filtration through a small pad of silica (elution with 10% EtOAc/hexane) to provide the desired compound (229 mg, 85.9%). LC/MS: (FA) ES− 419.

### Step 4 Preparation of tert-butyl 3-((1.5)-1-azido-2-{[tert-butyl(dimethyl)silyl]oxy}ethyl)-5-(trifluoromethyl)benzoate

A solution of tert-butyl 3-((1R)-2-{[tert-butyl(dimethyl)silyl]oxy}-1-hydroxyethyl)-5-(trifluoromethyl)benzoate (3) (229 mg, 0.54 mmol) in THF (5.0 mL), was cooled to 0° C. Triphenylphosphine (357 mg, 1.36 mmol) was added, followed by the dropwise addition of diisopropyl azodicarboxylate (268 uL, 1.36 mmol) and diphenylphosphonic azide (293 uL, 1.36 mmol). The reaction mixture was stirred at 0° C. for 2 h. The LC/MS showed consumption of the starting material. The solvents were evaporated and the residue was purified by chromatography on silica (elution with 2% to 10% EtOAc/hexane) to provide the desired compound as a clear oil (202 mg, 83.3%). ¹H NMR (400 MHz, CDCl₃): δ 8.17 (s, 1H), 8.09 (s, 1H), 7.76 (s, 1H), 4.70 (dd, 1H), 3.83 (m, 2H), 1.62 (s, 9H), 0.88 (s, 9H), 0.05 (s, 3H), and 0.03 (s, 3H).

### Step 5 Preparation of tert-butyl 3-((1S)-1-amino-2-{[tert-butyl(dimethyl)silyl]oxy}ethyl)-5-(trifluoromethyl)benzoate

To a solution of tert-butyl 3-((1S)-1-azido-2-{[tert-butyl(dimethyl)silyl]oxy}ethyl)-5-(trifluoromethyl)benzoate (202 mg, 0.45 mmol) in THF (4.00 mL) and water (81.7 uL, 4.50 mmol), was added triphenylphosphine (357 mg, 1.36 mmol). The reaction mixture was heated at 60° C. for 3 h. After this time, water (81.7 uL, 4.50 mmol) was added and heating continued overnight. The reaction mixture was cooled to rt and the solvents were evaporated. The residue was purified by chromatography on silica (elution with 12% to 50% EtOAc/hexane) to provide the desired product as a clear oil (136 mg, 71.5%). LC/MS: (FA) ES+ 420.3.

### Step 6 Preparation of 3-{(1S)-1-[(tert-butoxycarbonyl)amino]-2-hydroxyethyl}-5-(trifluoromethyl)benzoic acid

To a solution of tert-butyl 3-((1S)-1-amino-2-{[tert-butyl(dimethyl)silyl]oxy}ethyl)-5-(trifluoromethyl)benzoate (136 mg, 0.32 mmol) in DCM (3.00 mL) at 0° C. was added TFA (250 uL, 3.20 mmol) dropwise. The reaction mixture was allowed to warm slowly to rt and stirred for 48 h. LC/MS of the reaction mixture showed the desired product which was contaminated with approximately 40% 3-((1S)-1-amino-2-{[tert-butyl(dimethyl)silyl]oxy}ethyl)-5-(trifluoromethyl)benzoic acid. The solvents were evaporated. The residue was dissolved in dioxane (6.0 mL) and 1N NaOH solution (1.85 mL, 1.85 mmol) was added, followed by the addition of di-tert-butyldicarbonate (270 mg, 1.20 mmol). The reaction mixture was stirred at rt for 3 h. The solvents were evaporated and the residue was dissolved in EtOAc. 1N HCl was added to acidify the aqueous phase, and extracted into EtOAc (3×). The combined organic phases were washed with 1N HCl, then brine, dried (Na₂SO₄) and evaporated. The residue was purified by chromatography on silica (elution with 15% to 60% of (10% MeOH/1% AcOH/DCM)/DCM to provide the desired product (50.0 mg, 44.0%). LC/MS: (FA) ES− 348.2.

### Example 11

### 3-(1H-imidazol-5-yl)-5-(trifluoromethyl)benzoic acid

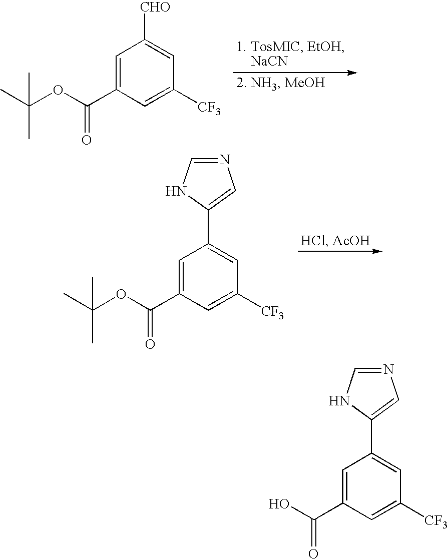

### Step 1 Preparation of tert-butyl 3-(1H-imidazol-5-yl)-5-(trifluoromethyl)benzoate

To a solution of tent-butyl 3-formyl-5-(trifluoromethyl)benzoate (1.05 g, 3.83 mmol) and p-tolylsulfonylmethyl isocyanide (748 mg, 3.83 mmol) in ethanol (10.0 mL), was added sodium cyanide (18.8 mg, 0.38 mmol). The reaction was stirred at rt for 1 h. LCMS showed the desired oxazoline intermediate. The solvents were evaporated and the residue was dissolved in 7.0 M ammonia in methanol solution (9.45 mL, 66.1 mmol) and the mixture was heated at 100° C. in a sealed tube for 3 h. After this time LCMS showed the desired product. The solvents were evaporated and the residue was purified by column chromatography (SiO₂, elution with 1.5-6% MeOH/DCM) to provide the desired compound as a beige solid (269 mg, 22.5%). LC/MS: (FA) ES+ 313, ES− 311.

### Step 2 Preparation of 3-(1H-imidazol-5-yl)-5-(trifluoromethyl)benzoic acid

tert-butyl 3-(1H-imidazol-5-yl)-5-(trifluoromethyl)benzoate (479 mg, 1.53 mmol) was dissolved in a mixture of acetic acid (5.00 mL) and concentrated hydrochloric acid (94.0 uL, 3.07 mmol) and the reaction mixture was stirred overnight. The solvents were evaporated and the residue was azeotroped with toluene and hexane, then triturated with EtOAc and dried under vacuum to provide the desired compound as a beige solid (397 mg, 100%). LC/MS: (FA) ES+ 257, ES− 255

### Example 12

### 3-(1-methyl-1H-imidazol-4-yl)-5-(trifluoromethyl)benzoic acid.HCl

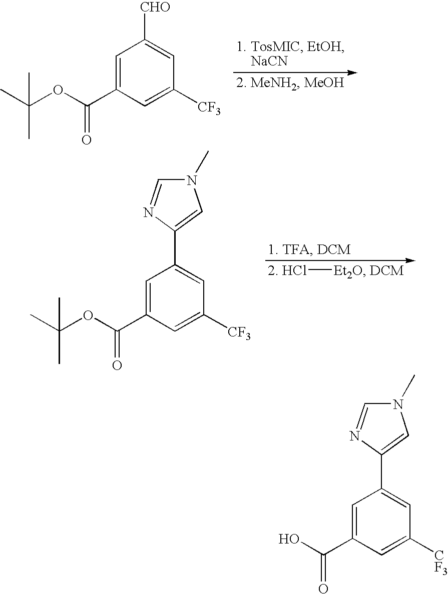

### Step 1 Preparation of tert-butyl 3-(1-methyl-1H-imidazol-4-yl)-5-(trifluoromethyl)benzoate

To a solution of tert-butyl 3-formyl-5-(trifluoromethyl)benzoate (4.00 g, 14.6 mmol) and p-tolylsulfonylmethyl isocyanide (2.85 g, 14.6 mmol) in ethanol (800 mL), was added sodium cyanide (71.5 mg, 1.46 mmol). The reaction was stirred at rt for 1 h. LCMS showed the desired oxazoline intermediate. The solvents were evaporated until 5-10 mL ethanol remained and the residue was dissolved in 2.0 M methylamine in methanol (36.5 mL, 72.9 mmol) and the mixture was heated at 75° C. in a sealed tube for 4 h. On cooling a precipitate formed which was filtered off. The filtrate was evaporated and the residue was purified by column chromatography (SiO₂, elution 1.2% to 5% MeOH/DCM). The material was further purified by column chromatography (SiO₂, elution 17% to 70% EtOAc/hexane) to provide the desired compound as a solid (262 mg, 5.5%). LC/MS: (FA) ES+ 327

### Step 2 Preparation of 3-(1-methyl-1H-imidazol-4-yl)-5-(trifluoromethyl)benzoic acid HCl

To a solution of tert-butyl 3-(1-methyl-1H-imidazol-4-yl)-5-(trifluoromethyl)benzoate in DCM (1.00 mL), was added TFA (1.21 mL). The reaction was stirred for 3 h. The solvents were evaporated and the residue was azeotroped with toluene. The residue was then treated with DCM and 2.0 M HCl-Et₂O solution and the solvents were evaporated to provide the desired compound as the HCl salt (254 mg, 100%). LC/MS: (FA) ES+ 271.

### Example 13

### Methyl 3-bromo-5-(trifluoromethyl)benzoate

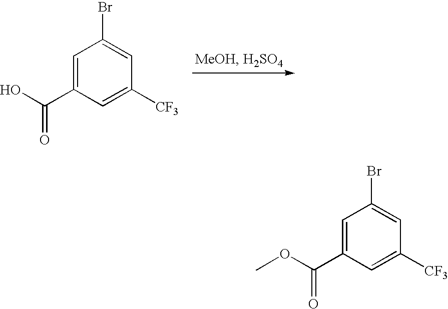

### Step 1 Preparation of methyl 3-bromo-5-(trifluoromethyl)benzoate

Sulfuric acid (7.90 mL, 148 mmol) was added dropwise to a solution of 3-bromo-5-(trifluoromethyl)benzoic acid (10.0 g, 37.2 mmol) in methanol (150 mL). The mixture was heated at 60° C. overnight. The methanol was evaporated and EtOAc was added. The mixture was basified by the addition of saturated NaHCO₃ solution and extracted into EtOAc (2×). The combined organic phases were washed with water and brine, dried (Na₂SO₄) and evaporated to provide the desired compound as an oil (10.5 g, 94.6%). ¹H NMR (400 MHz, CDCl₃): δ 8.36 (s, 1H), 8.23 (s, 1H), 7.95 (s, 1H), and 5.97 (s, 3H).

### Example 14

### 3-(1-methyl-1H-pyrazol-4-yl)-5-(trifluoromethyl)benzoic acid (Method 1)

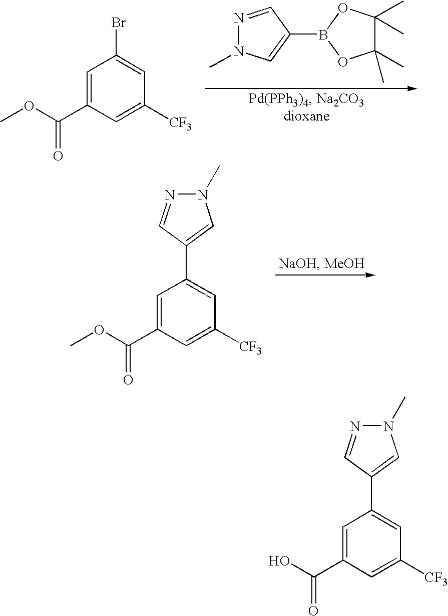

### Step 1 Preparation of methyl 3-(1-methyl-1H-pyrazol-4-yl)-5-(trifluoromethyl)benzoate

To a solution of methyl 3-bromo-5-(trifluoromethyl)benzoate (21.8 g, 77.0 mmol) in dioxane (218 mL), and water (131 mL), was added 1-methyl-4-(4,4,5,5-tetramethyl-1,3,2-dioxaborolan-2-yl)-1H-pyrazole (24.0 g, 116 mmol), sodium carbonate (27.7 g, 261 mmol) and tetrakis(triphenyphosphine)palladium(0) (4.40 g, 3.80 mmol). The reaction mixture was heated at 80° C. for 3 h. after which time TLC (20% EtOAc/hexane) showed consumption of the starting material. The reaction was cooled to rt and the precipitate was filtered off. The filtrate was diluted with water and extracted into EtOAc (2×). The organic phases were washed with brine, dried (Na₂SO₄) and evaporated. The residue was purified by filtration through silica, eluting with 0% to 40% EtOAc/hexane to provide the desired compound as a pale yellow solid (22.3 g, 100%). LC/MS: (FA) ES+ 285.

### Step 2 Preparation of 3-(1-methyl-1H-pyrazol-4-yl)-5-(trifluoromethyl)benzoic acid

To a solution of methyl 3-(1-methyl-1H-pyrazol-4-yl)-5-(trifluoromethyl)benzoate (22.3 g, 78.5 mmol) in methanol (375 mL), was added 1 N NaOH solution (314 mL, 314 mmol). The reaction was stirred at rt for 2 h. The methanol was evaporated and the aqueous residue was acidified to pH 2 with 1 N HCl. The precipitate was filtered off, washed with water and hexane and dried under vacuum to provide the desired compound as a white solid (20.3 g, 95.7%). LC/MS: (FA) ES+ 271, ES− 269.

3-(1H-pyrazol-4-yl)-5-(trifluoromethyl)benzoic acid was prepared from the appropriate starting materials in a method analogous to that described for 3-(1-methyl-1H-pyrazol-4-yl)-5-(trifluoromethyl)benzoic acid and the corresponding intermediates. LC/MS: (FA) ES+ 257.

### Example 15

### 3-(1-methyl-1H-pyrazol-5-yl)-5-(trifluoromethyl)benzoic acid

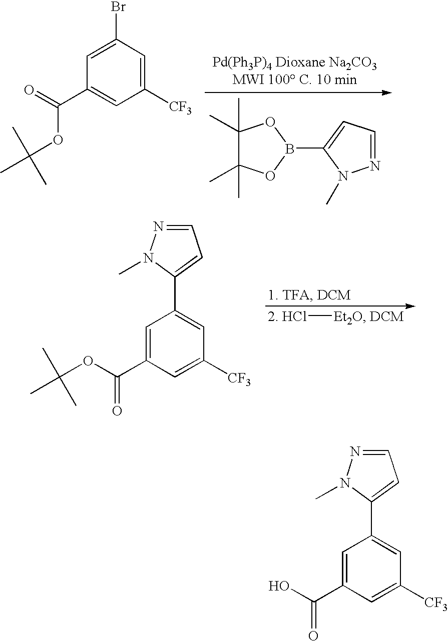

### Step 1 Preparation of tert-butyl 3-(1-methyl-1H-pyrazol-5-yl)-5-(trifluoromethyl)benzoate

tent-Butyl 3-bromo-5-(trifluoromethyl)benzoate (0.380 g, 1.17 mmol) and 1-methyl-5-(4,4,5,5-tetramethyl-1,3,2-dioxaborolan-2-yl)-1H-pyrazole (0.250 g, 1.20 mmol) were added to a microwave-safe vial. 1,4-Dioxane (2 mL) and 2.0 M sodium carbonate solution (1.71 mL, 3.42 mmol) were added. The reaction was degassed under an atmosphere of nitrogen for 3 minutes and tetrakis(triphenylphosphine)palladium(0) (0.270 g, 0.234 mmol) was added. The vial was then sealed and irradiated in the microwave at 100° C. for 10 minutes. The reaction vial was unsealed, and the mixture was diluted with ethyl acetate. The organic phase was washed with water and brine, dried over Na₂SO₄, filtered, and concentrated. Purification by column chromatography (SiO₂, elution with 0-10% ethyl acetate in dichloromethane) provided a 259 mg of a colorless oil (68% yield). LC/MS: (FA) ES+ 327.

### Step 2 Preparation of 3-(1-methyl-1H-pyrazol-5-yl)-5-(trifluoromethyl)benzoic acid

3-(1-methyl-1H-pyrazol-5-yl)-5-(trifluoromethyl)benzoic acid was prepared from the appropriate starting materials in a method analogous to that described for 3-(1-methyl-1H-imidazol-4-yl)-5-(trifluoromethyl)benzoic acid.HCl. LC/MS: (FA) ES+ 271.

3-(1H-pyrazol-5-yl)-5-(trifluoromethyl)benzoic acid was prepared from the appropriate starting materials in a method analogous to that described for 3-(1-methyl-1H-pyrazol-5-yl)-5-(trifluoromethyl)benzoic acid and the corresponding intermediates. LC/MS: (FA) ES+ 257.

### Example 16

### 3-[(1S)-1-amino-2-hydroxyethyl]-N-{(2R)-7-[(7-oxo-5,6,7,8-tetrahydro-1,8-naphthyridin-4-yl)oxy]-1,2,3,4-tetrahydronaphthalen-2-yl}-5-(trifluoromethyl)benzamide HCl salt

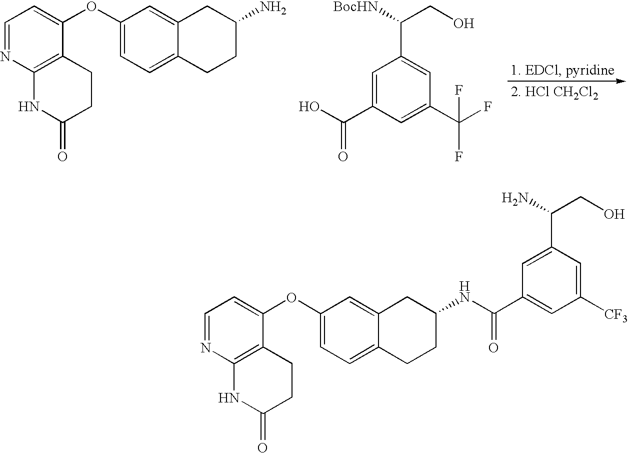

### Step 1 Preparation of tert-butyl {(1S)-2-hydroxy-1-[3-[({(2R)-7-[(7-oxo-5,6,7,8-tetrahydro-1,8-naphthyridin-4-yl)oxy]-1,2,3,4-tetrahydronaphthalen-2-yl}amino)carbonyl]-5-(trifluoromethyl)phenyl]ethyl}carbamate

To a solution of 3-{(1S)-1-[(tert-butoxycarbonyl)amino]-2-hydroxyethyl}-5-(trifluoromethyl)benzoic acid (24.0 mg, 0.07 mmol) and 5-{[(7R)-7-amino-5,6,7,8-tetrahydronaphthalen-2-yl]oxy}-3,4-dihydro-1,8-naphthyridin-2(1H)-one (21.2 mg, 0.07 mmol) in pyridine (1.00 mL) was added N-(3-dimethylaminopropyl)-N-ethylcarbodiimide hydrochloride (19.8 mg, 0.10 mmol). The reaction was stirred at rt overnight. The solvents were evaporated and the residue was purified by column chromatography (SiO₂, elution with 1.5% to 6% MeOH/DCM) to provide the desired compound as a white solid (21.0 mg, 48%). LC/MS: (FA) ES+ 641.34.

### Step 2 Preparation of 3-[(1S)-1-amino-2-hydroxyethyl]-N-{(2R)-7-[(7-oxo-5,6,7,8-tetrahydro-1,8-naphthyridin-4-yl)oxy]-1,2,3,4-tetrahydronaphthalen-2-yl}-5-(trifluoromethyl)benzamide.HCl salt

To a solution of tert-butyl {(1S)-2-hydroxy-1-[3-[({(2R)-7-[(7-oxo-5,6,7,8-tetrahydro-1,8-naphthyridin-4-yl)oxy]-1,2,3,4-tetrahydronaphthalen-2-yl}amino)carbonyl]-5-(trifluoromethyl)phenyl]ethyl}carbamate (0.021 g, 0.033 mmol) in DCM (1 mL) was added a solution of HCl in ether (2.0 M, 0.500 mL, 1.0 mmol). The reaction mixture was stirred at rt for 90 min and then the solvents were removed under reduced pressure. Purification by column chromatography (SiO₂, elution with 2.5-10% MeOH in DCM with 1% NH₄OH) provided a white solid. LC/MS: (FA) ES+ 541. ¹H NMR (400 MHz, d6 DMSO): δ 10.55 (s, 1H), 8.82 (d, 1H), 8.58 (br s, 2H), 8.36 (s, 1H), 8.25 (s, 1H), 8.08 (s, 1H), 7.98 (d, 1H), 7.21 (d, 1H), 6.93-6.90 (m, 2H), 6.32 (d, 1H), 4.56-4.49 (m, 1H), 4.25-4.20 (m, 1H), 3.83-3.73 (m, 2H), 3.10-3.05 (m, 1H), 2.94-2.80 (m, 5H), 2.54 (t, 2H), 2.12-2.05 (m, 1H), and 1.88-1.78 (m, 1H).

Compounds in the following table were prepared from the appropriate starting materials in a method analogous to that described for 3-[(1S)-1-amino-2-hydroxyethyl]-N-{(2R)-7-[(7-oxo-5,6,7,8-tetrahydro-1,8-naphthyridin-4-yl)oxy]-1,2,3,4-tetrahydronaphthalen-2-yl}-5-(trifluoromethyl)benzamide.HCl salt and the corresponding intermediates.

3-{[(2,2-difluoroethyl)amino]methyl}-N-{(2R)-7-[(7-oxo-5,6,7,8-tetrahydro-1,8-naphthyridin-4-
yl)oxy]-1,2,3,4-tetrahydronaphthalen-2-yl}-5-(trifluoromethyl)benzamide
¹H NMR (300 MHz, d6 DMSO): δ 10.65 (br s, 1H), 10.10 (br s, 1H), 8.8 5(d, 1H), 8.45 (br s, 1H),
8.3 (br s, 1H), 8.20 (br s, 1H), 8.0 (br s, 1H), 7.20 (d, 1H), 6.95(m, 2H), 6.39 (m, 1H), 4.40 (br s,
1H), ), 4.20 (m, 2H), 3.60 (m, 2H), 3.40 (m, 1H), 3.07 (m, 1H), 2.90 (m, 4H), 2.10 (m, 1H), 1.85 (m,
1H), and 1.10 (m, 1H).
3-(1-amino-1-methylethyl)-N-{(2R)-7-[(7-oxo-5,6,7,8-tetrahydro-1,8-naphthyridin-4-yl)oxy]-
1,2,3,4-tetrahydronaphthalen-2-yl}-5-(trifluoromethyl)benzamide
¹H NMR (400 MHz, d6 DMSO, 2 HCl salt): δ 10.77 (s, 1H), 8.98-8.96 (m, 4H), 8.53 (s, 1H), 8.20 (s,
1H), 8.10 (s, 1H), 8.03 (d, 1H), 7.22 (d, 1H), 6.94-6.91 (m, 2H), 6.39 (d, 1H), 4.21 (br s, 1H), 3.11-
3.04 (m, 1H), 2.96-2.86 (m, 5H), 2.56 (t, 2H), 2.10-2.05 (m, 1H), 1.90-1.80 (m, 1H), and 1.70 (s, 6H).
3-(aminomethyl)-N-{(2R)-7-[(7-oxo-5,6,7,8-tetrahydro-1,8-naphthyridin-4-yl)oxy]-1,2,3,4-
tetrahydronaphthalen-2-yl}-5-(trifluoromethyl)benzamide
¹H NMR (400 MHz, d6 DMSO, 2 HCl salt): δ 10.60 (s, 1H), 8.83 (d, 1H), 8.46 (br s, 2H), 8.37 (s,
1H), 8.24 (s, 1H), 8.08 (s, 1H), 7.99 (d, 1H), 7.21 (d, 1H), 6.95-6.91 (m, 2H), 6.34 (d, 1H), 4.24-4.18
(m, 3H), 3.11-3.05 (m, 1H), 2.94-2.81 (m, 5H), 2.54 (t, 2H), 2.09-2.04 (m, 1H), and 1.88-1.79 (m,
1H).
3-[(dimethylamino)methyl]-N-{(2R)-7-[(7-oxo-5,6,7,8-tetrahydro-1,8-naphthyridin-4-yl)oxy]-
1,2,3,4-tetrahydronaphthalen-2-yl}-5-(trifluoromethyl)benzamide
¹H NMR (400 MHz, d6 DMSO, 2 HCl salt) δ 10.67 (s, 1H), 8.86 (d, 1H), 8.43 (s, 1H), 8.31 (s, 1H),
8.20 (s, 1H), 8.00 (d, 1H), 7.22 (d, 1H), 6.91-6.96 (m, 2H), 6.36 (d, 1H), 4.42-4.49 (m, 2H), 4.17-4.28
(m, 1H), 3.04-3.12 (m, 1H), 2.81-2.97 (m, 5H), 2.69-2.75 (m, 6H), 2.53-2.59 (m, 2H), 2.03-2.14 (m,
1H), and 1.77-1.89 (m, 1H).
3-[(methylamino)methyl]-N-{(2R)-7-[(7-oxo-5,6,7,8-tetrahydro-1,8-naphthyridin-4-yl)oxy]-
1,2,3,4-tetrahydronaphthalen-2-yl}-5-(trifluoromethyl)benzamide
¹H NMR (400 MHz, d6 DMSO, 2 HCl salt): δ 9.26-9.36 (m, 2H), 8.85 (d, 1H), 8.40 (s, 1H), 8.27 (s,
1H), 8.13 (s, 1H), 8.00 (d, 1H), 7.20-7.24 (m, 1H), 6.90-6.94 (m, 2H), 6.35 (d, 1H), 4.18-4.31 (m, 3H),
3.04-3.11 (m, 1H), 2.80-2.95 (m, 5H), 2.53-2.59 (m, 5H), 2.04-2.11 (m, 1H), and 1.79-1.88 (m, 1H).
3-(1-aminocyclobutyl)-N-{(2R)-7-[(7-oxo-5,6,7,8-tetrahydro-1,8-naphthyridin-4-yl)oxy]-1,2,3,4-
tetrahydronaphthalen-2-yl}-5-(trifluoromethyl)benzamide
¹H NMR (400 MHz, d6 DMSO, 2 HCl salt): δ 10.60 (s, 1H), 8.92 (d, 1H), 8.85 (br s, 2H), 8.40 (s,
1H), 8.25 (s, 1H), 8.02 (s, 1H), 7.99 (d, 1H), 7.22 (d, 1H), 6.93-6.91 (m, 2H), 6.34 (d, 1H), 4.22 (br s,
1H), 3.11-3.05 (m, 1H), 2.94-2.83 (m, 5H), 2.75-2.58 (m, 4H), 2.54 (t, 2H), 2.28-2.19 (m, 1H), 2.11-
2.06 (m, 1H), and 1.88-1.78 (m, 2H).
3-{[3-(hydroxymethyl)morpholin-4-yl]methyl}-N-{(2R)-7-[(7-oxo-5,6,7,8-tetrahydro-1,8-
naphthyridin-4-yl)oxy]-1,2,3,4-tetranydronaphthalen-2-yl}-5-(trifluoromethyl)benzamide
¹H NMR (300 MHz, d6 DMSO): δ 10.47 (s, 1H), 8.69 (d, 1H), 8.08 (s, 2H), 7.95 (d, 1H), 7.83 (s,
1H), 7.20 (d, 1H), 6.91 (s, 2H), 6.30 (d, 1H), 4.64 (s, 1H), 4.18 (d, 2H), 3.75 (d, 1H), 3.68-3.54 (m,
2H), 3.47-3.32 (m, 6H), 3.07 (d, 1H), 2.92-2.82 (m, 5H), 2.52 (d, 1H), 2.24-2.05 (m, 2H), and 1.86-
1.73 (m, 1H).
3-[(ethylamino)methyl]-N-{(2R)-7-[(7-oxo-5,6,7,8-tetrahydro-1,8-naphthyridin-4-yl)oxy]-1,2,3,4-
tetrahydronaphthalen-2-yl}-5-(trifluoromethyl)benzamide
¹H NMR (400 MHz, CD₃OD, 2HCl salt): δ 8.37 (s, 1H), 8.26 (s, 1H), 8.11 (d, 1H), 8.07 (s, 1H), 7.32
(d, 1H), 7.05-6.99 (m, 2H), 6.73 (d, 1H), 4.41-4.31 (m, 3H), 3.25-3.15 (m, 5H), 3.06-2.92 (m, 3H),
2.82 (t, 2H), 2.27-2.19 (m, 1H), 2.02-1.9 (m, 1H), and 1.37 (t, 3H).
3-(1-methyl-1H-pyrazol-4-yl)-N-{(2R)-7-[(7-oxo-5,6,7,8-tetrahydro-1,8-naphthyridin-4-yl)oxy]-
1,2,3,4-tetrahydronaphthalen-2-yl}-5-(trifluoromethyl)benzamide
¹H NMR (400 MHz, d6 DMSO, HCl salt): δ: 10.56 (s, 1H), 8.74 (d, 1H), 8.40 (s, 1H), 8.31 (s, 1H),
8.09-8.07 (m, 2H), 7.98-7.97 (m, 2H), 7.21 (d, 1H), 6.94-6.90 (m, 2H), 6.33 (d, 1H), 4.22 (br s, 1H),
3.89 (s, 3H), 3.13-3.06 (m, 1H), 2.94-2.80 (m, 5H), 2.54 (t, 2H), 2.13-2.06 (m, 1H), and 1.88-1.79 (m,
1H).
N-{(2R)-7-[(7-oxo-5,6,7,8-tetrahydro-1,8-naphthyridin-4-yl)oxy]-1,2,3,4-tetrahydronaphthalen-2-
yl}-3-(1H-pyrazol-4-yl)-5-(trifluoromethyl)benzamide
¹H NMR (400 MHz, d6 DMSO, HCl salt): δ 10.61 (s, 1H), 8.75 (d, 1H), 8.37 (s, 1H), 8.31 (s, 2H),
8.12 (s, 1H), 7.99-7.98 (m, 2H), 7.22 (d, 1H), 6.94-6.91 (m, 2H), 6.35 (d, 1H), 4.22 (br s, 1H), 3.12-
3.07 (m, 1H), 2.95-2.81 (m, 5H), 2.55 (t, 2H), 2.14-2.07 (m, 1H), and 1.87-1.78 (m, 1H).
3-(1-methyl-1H-imidazol-4-yl)-N-{(2R)-7-[(7-oxo-5,6,7,8-tetrahydro-1,8-naphthyridin-4-yl)oxy]-
1,2,3,4-tetrahydronaphthalen-2-yl}-5-(trifluoromethyl)benzamide
¹H NMR (400 MHz, d6 DMSO, 2 HCl salt): δ: 10.63 (s, 1H), 9.23 (s, 1H), 8.95 (d, 1H), 8.75 (s, 1H),
8.48 (s, 1H), 8.43 (s, 1H), 8.26 (s, 1H), 7.99 (d, 1H), 7.22 (d, 1H), 6.94-6.91 (m, 2H), 6.35 (d, 1H),
4.25 (br s, 1H), 3.91 (s, 3H), 3.12-3.06 (m, 1H), 2.96-2.85 (m, 5H), 2.55 (t, 2H), 2.13-2.07 (m, 1H),
and 1.91-1.81 (m, 1H).
3-(1H-imidazol-4-yl)-N-{(2R)-7-[(7-oxo-5,6,7,8-tetrahydro-1,8-naphthyridin-4-yl)oxy]-1,2,3,4-
tetrahydronaphthalen-2-yl}-5-(trifluoromethyl)benzamide
¹H NMR (400 MHz, d6 DMSO, 2 HCl salt): δ 10.53 (s, 1H), 9.25 (s, 1H), 8.90 (d, 1H), 8.73 (s, 1H),
8.47 (s, 1H), 8.43 (s, 1H), 8.25 (s, 1H), 7.98 (d, 1H), 7.22 (d, 1H), 6.93-6.90 (m, 2H), 6.32 (d, 1H),
4.25 (br s, 1H), 3.13-3.07 (m, 1H), 2.93-2.84 (m, 5H), 2.54 (t, 2H), 2.12-2.08 (m, 1H), and 2.91-2.81
(m, 1H).
3-(1-methyl-1H-pyrazol-5-yl)-N-{(2R)-7-[(7-oxo-5,6,7,8-tetrahydro-1,8-naphthyridin-4-yl)oxy]-
1,2,3,4-tetrahydronaphthalen-2-yl}-5-(trifluoromethyl)benzamide
¹H NMR (400 MHz, CD₃OD, HCl salt): δ 8.25 (s, 1H), 8.23 (s, 1H), 7.98 (s, 1H), 7.93 (d, 1H), 7.56
(d, 1H), 7.22 (d, 1H), 6.88-6.91 (m, 2H), 6.54 (d, 1H), 6.35 (d, 1H), 4.32-4.40 (m, 1H), 3.92 (s, 3H),
3.15-3.20 (m, 1H), 2.98-3.07 (m, 4H), 2.85-2.92 (m, 1H), 2.66-2.68 (m, 2H), 2.19-2.26 (m, 1H), and
1.86-1.96 (m, 1H).
N-{(2R)-7-[(7-oxo-5,6,7,8-tetrahydro-1,8-naphthyridin-4-yl)oxy]-1,2,3,4-tetrahydronaphthalen-2-
yl}-3-(1H-pyrazol-5-yl)-5-(trifluoromethyl)benzamide
¹H NMR (400 MHz, CD₃OD, HCl salt): δ 8.53 (s, 1H), 8.28 (s, 1H), 8.09 (s, 1H), 7.94 (d, 1H), 7.77
(d, 1H), 7.22 (d, 1H), 6.86-6.90 (m, 3H), 6.36 (d, 1H), 4.32-4.40 (m, 1H), 3.16-3.21 (m, 1H), 2.99-
3.07 (m, 4H), 2.87-2.93 (m, 1H), 2.64-2.68 (m, 2H), 2.20-2.26 (m, 1H), and 1.86-1.98 (m, 1H).

3-(3-hydroxyazetidin-3-yl)-N-{(2R)-7-[(7-oxo-5,6,7,8-tetrahydro-1,8-naphthyridin-4-yl)oxy]-1,2,3,4-tetrahydronaphthalen-2-yl}-5-(trifluoromethyl)benzamide was prepared from the appropriate starting materials in a method analogous to that described for the preparation of tert3-[(1S)-1-amino-2-hydroxyethyl]-N-{(2R)-7-[(7-oxo-5,6,7,8-tetrahydro-1,8-naphthyridin-4-yl)oxy]-1,2,3,4-tetrahydronaphthalen-2-yl}-5-(trifluoromethyl)benzamide HCl salt (step 1) and the corresponding intermediates, followed by standard deprotection and HCl salt formation conditions. ¹H NMR (300 MHz, d6 DMSO, 2 HCl salt): δ 10.55 (s, 1H), 9.40 (br s, 1H), 9.25 (br s, 1H), 8.92 (d, 1H), 8.52 (s, 1H), 8.21 (s, 1H), 8.07 (s, 1H), 7.98 (d, 1H), 7.33 (d, 1H), 6.93-6.90 (m, 2H), 6.33 (d, 1H), 4.49-4.40 (m, 2H), 4.29-4.11 (m, 3H), 3.11-3.03 (m, 1H), 2.95-2.85 (m, 5H), 2.54 (t, 2H), 2.13-2.04 (m, 1H), and 1.93-1.81 (m, 1H).

While the foregoing invention has been described in some detail for purposes of clarity and understanding, these particular embodiments are to be considered as illustrative and not restrictive. It will be appreciated by one skilled in the art from a reading of this disclosure that various changes in form and detail can be made without departing from the true scope of the invention, which is to be defined by the appended claims rather than by the specific embodiments.

The patent and scientific literature referred to herein establishes knowledge that is available to those with skill in the art. Unless otherwise defined, all technical and scientific terms used herein have the same meaning as commonly understood by one of ordinary skill in the art to which this invention belongs. The issued patents, applications, and references that are cited herein are hereby incorporated by reference to the same extent as if each was specifically and individually indicated to be incorporated by reference. In the case of inconsistencies, the present disclosure, including definitions, will control.

What is claimed is:

1. A process for preparing a compound of formula (I): 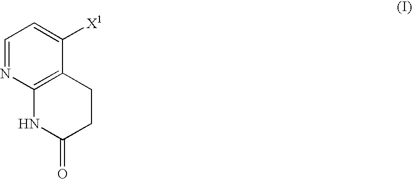
wherein X¹ is Cl or F;
the process comprising coupling a compound of formula (II): 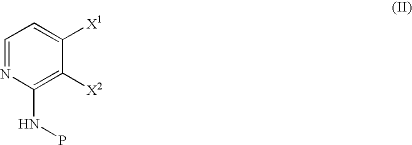
wherein:
X¹ is Cl or F;
X² is Br or I; and
P is hydrogen or an amino group protecting moiety that is labile to the reaction conditions;
with a compound of formula (III): 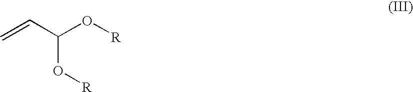
wherein each R independently is C₁₋₄ alkyl, —C(O)—(C₁₋₄ alkyl), C₆₋₁₀ ar(C₁₋₄)alkyl, or —C(O)—(C₆₋₁₀ ar(C₁₋₄)alkyl), where the aryl portion of any such groups is substituted or unsubstituted;
in a reaction mixture comprising a palladium catalyst and a base, to form a compound of formula (I).

2. The process of claim 1, wherein the palladium catalyst is selected from the group consisting of palladium(II) chloride, palladium(II) acetate, tris(dibenzylideneacetone)-dipalladium, tetrakis(triphenylphosphine)palladium, bis(triphenylphosphine)palladium dichloride, (1,1′-bis(diphenylphosphino)ferrocene)palladium dichloride, di-chlorobis[5-chloro-2-[(4-chlorophenyl)(hydroxyimino)methyl]phenyl-C]di-palladium, trans-di-μ-acetobis [2-(di-o-tolylphosphino)benzyl]dipalladium.

3. The process of claim 1, wherein the reaction mixture further comprises an added phosphine ligand.

4. The process of claim 2, wherein the phosphine ligand is selected from the group consisting of triphenylphosphine, tri(o-tolyl)phosphine, tri(tert-butyl)phosphine, tri(2-furyl)-phosphine, 1,1′-bis(diphenylphosphino)ferrocene, 1,1′-bis(diphenylphosphino)methane, 1,1′-bis(diphenylphosphino)ethane, and 2-dicyclohexylphosphino-2′,4′,6′-triisopropylbiphenyl.

5. The process of claim 1, wherein each R is ethyl.

6. The process of claim 1, wherein the base is selected from the group consisting of potassium carbonate, cesium carbonate, sodium carbonate, sodium bicarbonate, sodium hydroxide, potassium hydroxide, lithium hydroxide, sodium acetate, and potassium acetate.

7. The process of claim 1, wherein the base is a tertiary amine base.

8. The process of claim 7, wherein the tertiary amine base is selected from the group consisting of triethylamine, diisopropylethylamine, dicyclohexylmethylamine, and 1,8-diazabicyclo[5.4.0]undec-7-ene.

9. The process of claim 1, wherein the reaction mixture comprises a solvent comprising dimethylformamide, dimethylacetamide, N-methylpyrrolidone, 1,4-dioxane, tert-butanol, or a mixture or aqueous mixture thereof.

10. The process of claim 9, wherein the solvent comprises dimethylformamide-water or dimethylacetamide-water.

11. The process of claim 10, wherein P is tert-butoxycarbonyl.

12. The process of claim 1, wherein the process further comprises preparing the compound of formula (II) by the steps:
(aa) treating a compound of formula (IV): 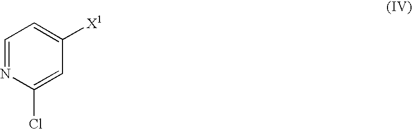
wherein X¹ is Cl or F;
with a compound of formula P¹—NH₂, wherein P¹ is an amino group protecting moiety, in a reaction mixture comprising a palladium catalyst and a base to form a compound of formula (V): 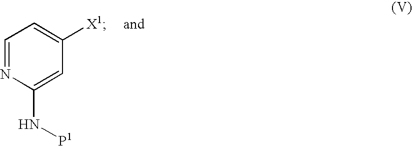
(bb) halogenating the compound of formula (V) to form the compound of formula (II), wherein P is an amino group protecting moiety.

13. The process of claim 12, further comprising the step:
(cc) removing the protecting group P¹ to form the compound of formula (II), wherein P is hydrogen.

14. The process of claim 12, wherein the palladium catalyst in step (aa) is selected from the group consisting of palladium(II) chloride, palladium(II) acetate, tris(dibenzylideneacetone)dipalladium, tetrakis(triphenylphosphine)palladium, bis(triphenylphosphine)palladium dichloride, and (1,1′-bis(diphenylphosphino)ferrocene) palladium dichloride.

15. The process of claim 12, wherein the reaction mixture in step (aa) further comprises an added phosphine ligand.

16. The process of claim 15, wherein the phosphine ligand is selected from the group consisting of tri(o-tolyl)phosphine, triphenylphosphine, tri(2-furyl)phosphine, 1,1′-bis(diphenylphosphino)ferrocene, 1,1′-bis(diphenylphosphino)methane, 1,1′-bis(diphenylphosphino)ethane, (oxydi-2,1-phenylene)bis(diphenylphosphine), 4,5-bis(diphenylphosphino)-9,9-dimethylxanthene, 2-dicyclohexylphosphino-2′,6′-dimethoxybiphenyl, and 2-dicyclohexylphosphino-2′,4′,6′-triisopropylbiphenyl.

17. The process of claim 16, wherein the palladium catalyst is palladium(II) acetate, and the phosphine ligand is 4,5-bis(diphenylphosphino)-9,9-dimethylxanthene, 2-dicyclohexylphosphino-2′,4′,6′-triisopropylbiphenyl, or (oxydi-2,1-phenylene)-bis(diphenylphosphine).

18. The process of claim 12, wherein the palladium catalyst is palladium(II) acetate, and the reaction mixture does not comprise an added phosphine ligand.

19. The process of claim 12, wherein the base is selected from the group consisting of potassium carbonate, cesium carbonate, sodium carbonate, sodium bicarbonate, sodium tert-butoxide, potassium tert-butoxide, sodium hydroxide, potassium hydroxide, and lithium hydroxide.

20. The process of claim 12, wherein halogenating step (bb) comprises treating the compound of formula (V) with a base and iodine to form the compound of formula (II), wherein X² is I, and P is an amino group protecting moiety.

21. The process of claim 20, wherein the base is an organolithium, an organomagnesium, or a silver salt.

22. The process of claim 12, wherein the halogenating step (bb) comprises treating the compound of formula (V) with a base and a brominating reagent to form the compound of formula (II), wherein X² is Br, and P is an amino group protecting moiety, wherein the brominating reagent is selected from the group consisting of ethylene bromide, N-bromosuccinimide, and bromine.

23. The process of claim 22, wherein the base is an organolithium, an organomagnesium, or a silver salt.

24. The process of claim 12, wherein the halogenating step (bb) comprises treating the compound of formula (V) with a tertiary amine and bromine to form the compound of formula (II), wherein X² is Br, and P is an amino group protecting moiety.

25. A process for preparing a compound of formula (VI): 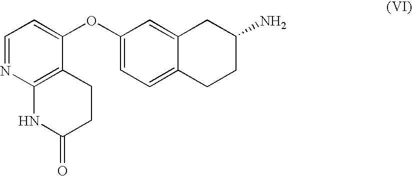
the process comprising
(i) coupling a compound of formula (I): 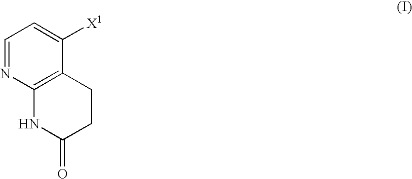
wherein X¹ is Cl or F;
with a compound of formula (VII): 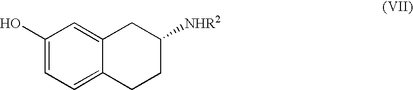
wherein R² is hydrogen, an amino group protecting moiety, or an acid addition salt; to form the compound of formula (VI-A): 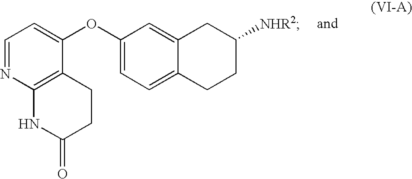
(ii) when R² is an amino group protecting moiety, removing the amino group protecting moiety to form the compound of formula (VI).

26. The process of claim 25, wherein steps (i) and (ii) occur in the same reaction mixture.

27. The process of claim 25, wherein the coupling is conducted in a reaction mixture comprising a base and a high-boiling polar solvent.

28. The process of claim 27, wherein the reaction mixture comprises Cs₂CO₃ and dimethylformamide.

29. The process of claim 25, wherein R² is hydrogen, tert-butoxycarbonyl, or H.HBr.

30. The process of claim 25, further comprising the step:
(iii) condensing the compound of formula (VI) with a compound of formula (VIII): 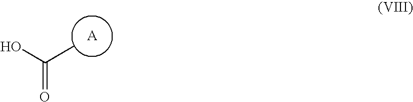
wherein Ring A is a substituted or unsubstituted phenyl ring, to form a compound of formula (IX):

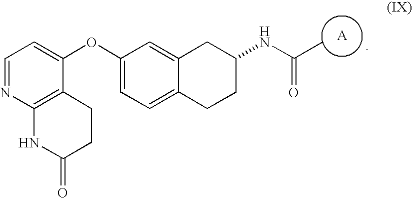

31. The process of claim 30, wherein the compound of formula (VIII) is characterized by formula (VIII-A): 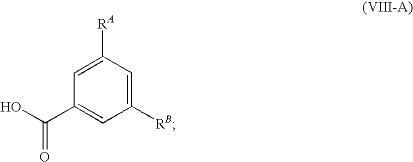
and the compound of formula (IX) is characterized by formula (IX-A): 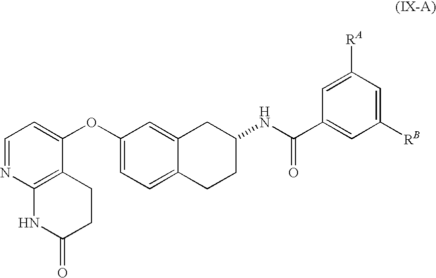
wherein:
R^{A} is halo, —CN, —CHO, —C(R^{5x})═C(R^{5x})(R^{5y}), —C≡C—R^{5y}, —OR^{5z}, —SR^{6x}, —N(R^{4y})(R^{4z}), —CO₂R^{6x}, —C(O)N(R^{4x})(R^{4y}); or R^{A} is a C₁₋₆ aliphatic or C₁₋₆ fluoroaliphatic optionally substituted with one or two substituents independently selected from the group consisting of —OR^{5x}, —N(R^{4y})(R^{4z}), —SR^{6x}, —CO₂R^{6x}, or —C(O)N(R^{4x})(R^{4y}); or R^{A} is an optionally substituted 5- or 6-membered nitrogen-containing heterocyclyl or heteroaryl ring;
R^{B} is selected from the group consisting of C₁₋₄ aliphatic, C₁₋₄ fluoroaliphatic, —O(C₁₋₄ aliphatic), —O(C₁₋₄ fluoroaliphatic), and halo; and
R^{4x} is hydrogen, C₁₋₄, aliphatic, C₁₋₄ fluoroaliphatic, or C₆₋₁₀ ar(C₁₋₄)alkyl, the aryl portion of which may be optionally substituted;
R^{4y} is hydrogen, C₆₋₁₀ ar(C₁₋₄)alkyl, the aryl portion of which may be optionally substituted, an optionally substituted 5- or 6-membered aryl, heteroaryl, or heterocyclyl ring, or a C₁₋₄ aliphatic or C₁₋₄ fluoroaliphatic optionally substituted with one or two substituents independently selected from the group consisting of —OR^{5x}, —N(R^{4x})₂, —CO₂R^{5x}, or —C(O)N(R^{4x})₂;
R^{4x} is an amino group protecting moiety, C₁₋₄ aliphatic, C₁₋₄ fluoroaliphatic, or C₆₋₁₀ ar(C₁₋₄)alkyl, the aryl portion of which may be optionally substituted; or
R^{4x} and R^{4y}, taken together with the nitrogen atom to which they are attached, form an optionally substituted 4- to 8-membered heterocyclyl or 5-membered heteroaryl ring having, in addition to the nitrogen atom, 0-2 ring heteroatoms independently selected from N, O, and S; or
R^{4y} and R^{4z}, taken together with the nitrogen atom to which they are attached, form an optionally substituted 4- to 8-membered heterocyclyl or 5-membered heteroaryl ring having, in addition to the nitrogen atom, 0-2 ring heteroatoms independently selected from N, O, and S;
each R^{5x} independently is hydrogen, C₁₋₄ aliphatic, C₁₋₄ fluoroaliphatic, or C₆₋₁₀ ar(C₁₋₄)alkyl, the aryl portion of which may be optionally substituted, or an optionally substituted 5- or 6-membered aryl, heteroaryl, or heterocyclyl ring;
each R^{5y} independently is hydrogen, an optionally substituted monocyclic nitrogen-containing heterocyclyl, an optionally substituted C₆₋₁₀ aryl, a C₆₋₁₀ar(C₁₋₄)alkyl, the aryl portion of which is optionally substituted, or a C₁₋₄ aliphatic or C₁₋₄ fluoroaliphatic optionally substituted with one or two substituents independently selected from the group consisting of —OR^{5x}, —N(R^{4x})₂, —CO₂R^{5x}, or —C(O)N(R^{4x})₂;
each R^{5z} independently is hydrogen, a hydroxy group protecting moiety, an optionally substituted monocyclic nitrogen-containing heterocyclyl, an optionally substituted C₆₋₁₀ aryl, a C₆₋₁₀ar(C₁₋₄)alkyl, the aryl portion of which is optionally substituted, or a C₁₋₄ aliphatic or C₁₋₄ fluoroaliphatic optionally substituted with one or two substituents independently selected from the group consisting of —OR^{5z}, —N(R^{4x})(R^{4y}), —CO₂R^{6x}, or —C(O)N(R^{4x})(R^{4y}); and
each R^{6x} independently is C₁₋₄ aliphatic, C₁₋₄ fluoroaliphatic, or C₆₋₁₀ ar(c)alkyl, the aryl portion of which may be optionally substituted.

32. The process of claim 31, wherein R^{A} is a substituted or unsubstituted pyrazolyl, oxazolyl, isoxazolyl, imidazolyl, triazolyl, or tetrazolyl ring.

33. The process of claim 32, wherein:
each substitutable ring carbon atom in R^{A} independently is unsubstituted or is substituted with halo, —OR^{5x}, —N(R^{4x})(R^{4y}), —N(R^{4x})—C(O)—R⁵, —C(O)—N(R^{4x})(R^{4y}), or a C₁₋₄ aliphatic or C₁₋₄ fluoroaliphatic group optionally substituted with ═O, —OR^{5x}, —N(R^{4x})(R^{4y}), —N(R^{4x})—C(O)—R⁵, or —C(O)—N(R^{4x})(R^{4y});
each substitutable ring nitrogen atom in R^{A} is unsubstituted or is substituted with —C(O)—R⁵, —C(O)N(R^{5x})₂, —SO₂R⁵, or a C₁₋₄ aliphatic or C₁₋₄ fluoroaliphatic group optionally substituted with ═O, —OR^{5x}, —N(R^{4x})(R^{4y}), —N(R^{4x})—C(O)—R⁵, or —C(O)—N(R^{4x})(R⁴).

34. The process of claim 31, wherein:
R^{A} has the formula —C(R^{a})(R^{b})—N(R^{c})(R^{d});
R^{a} is hydrogen, C₁₋₄ aliphatic, C₁₋₄ fluoroaliphatic, or -T¹-R²; or R^{a}, taken together with R^{b} and the carbon atom to which they are attached, forms a substituted or unsubstituted 3- to 6-membered carbocyclic ring; or R^{a}, taken together with R^{c} and the intervening carbon and nitrogen atoms, form a substituted or unsubstituted 4- to 6-membered heterocyclic ring;
R^{b} is hydrogen, C₁₋₄ aliphatic, or C₁₋₄ fluoroaliphatic; or R^{b}, taken together with R^{a} and the carbon atom to which they are attached, forms a substituted or unsubstituted 3- to 6-membered carbocyclic ring;
R^{c} is hydrogen, C₁₋₄ aliphatic, C₁₋₄ fluoroaliphatic, or -T¹-R²; or R^{c}, taken together with R^{a} and the intervening carbon and nitrogen atoms, form a substituted or unsubstituted 4- to 6-membered heterocyclic ring; or R^{c}, taken together with R^{d} and the nitrogen atom to which they are attached, forms a substituted or unsubstituted 3- to 6-membered heterocyclic ring or 5- to 6-membered heteroaryl ring;
R^{d} is an amino group protecting moiety, C₁₋₄ aliphatic, or C₁₋₄ fluoroaliphatic, or -T¹-R²; or R^{d}, taken together with R^{c} and the nitrogen atom to which they are attached, forms a substituted or unsubstituted 3- to 6-membered heterocyclic ring or 5- to 6-membered heteroaryl ring;
T¹ is a C₁₋₃ alkylene chain; and
R² is —OR^{5z}, —N(R^{4y})(R^{4z}), —N(R^{4x})—C(O)—R^{5x}, or —C(O)—N(R^{4x})(R^{4y}).

35. The process of claim 30, wherein the compound of formula (VIII) is characterized by formula (VIII-B): 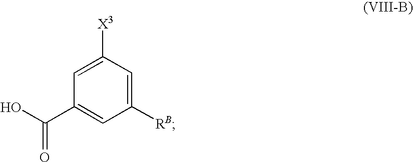
and the compound of formula (IX) is characterized by formula (IX-B): 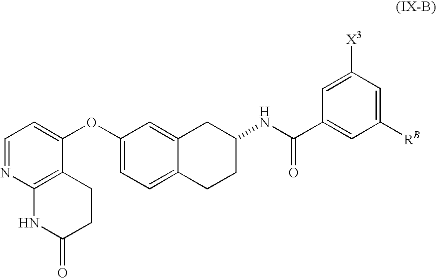
wherein:
X³ is Br or I; and
R^{B} is selected from the group consisting of Cl, C₁₋₄ aliphatic, C₁₋₄ fluoroaliphatic, —O(C₁₋₄ aliphatic), and —O(C₁₋₄ fluoroaliphatic).

36. The process of claim 35, further comprising the step:
(iv-a) coupling the compound of formula (IX-B) with a compound of formula (X): 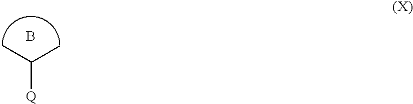
wherein Ring B is a substituted or unsubstituted aryl or heteroaryl ring; and
Q is a moiety selected from the group consisting of boronic acid, zinc halide, and trialkyltin;
in a reaction mixture comprising a palladium catalyst, to form a compound of formula (IX-C):

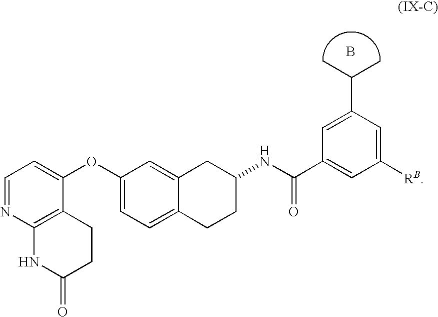

37. The process of claim 35, further comprising the step
(iv-b) coupling the compound of formula (IX-B) with a compound of formula (XI): 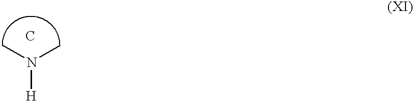
wherein Ring C is a substituted or unsubstituted heteroaryl ring;
in a reaction mixture comprising a palladium catalyst, to form a compound of formula (IX-D):

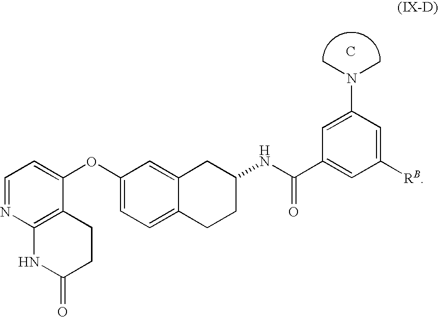

38. The process of claim 30, wherein the compound of formula (VIII) is characterized by formula (VIII-C): 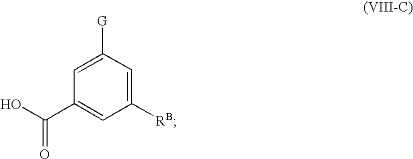
and the compound of formula (IX) is characterized by formula (IX-C): 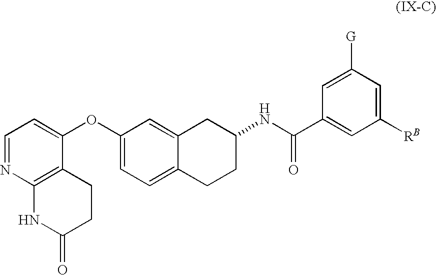
wherein:
G is —CN or —CHO; and
R³ is selected from the group consisting of Cl, C₁₋₄ aliphatic, C₁₋₄ fluoroaliphatic, —O(C₁₋₄ aliphatic), and —O(C₁₋₄ fluoroaliphatic).

39. The process of claim 38, wherein G is —CN, and the process further comprises treating the compound of formula (IX-C) with sodium azide to form a compound of formula (IX), wherein R^{A} is tetrazolyl.

40. The process of claim 38, wherein G is —CHO, and the process further comprises treating the compound of formula (IX-C) with p-tolylsulfonylmethyl isocyanide to form a compound of formula (IX), wherein R^{A} is 1,3-oxazol-5-yl.

41. The process of claim 38, wherein G is —CHO, and the process further comprises treating the compound of formula (IX-C) with p-tolylsulfonylmethyl isocyanide, followed by an amine, to form a compound of formula (IX), wherein R^{A} is imidazolyl.

42. A compound of formula (I) or a salt thereof: 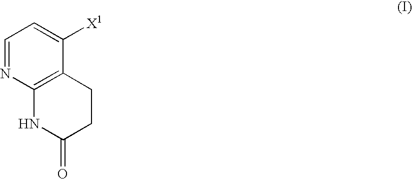
wherein X¹ is Cl or F.

43. A compound of formula (II) or a salt thereof: 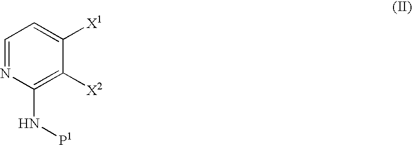
wherein X¹ is Cl or F, X² is Br or I, and P¹ is hydrogen or an amino group protecting moiety.

44. A compound of formula (VI-A) or a salt thereof: 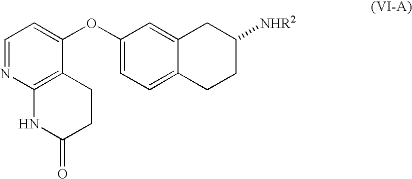
wherein R² is hydrogen, an amino group protecting moiety, or an acid addition salt.
